(19) United States
(12) Patent Application Publication
(10) Pub. No.: US 2026/0305663 A1
(43) Pub. Date: Oct. 8, 2026

(54) ADAPTOGENIC, HIGH-PROTEIN FOOD PRODUCT AND METHOD OF MAKING SAME

(71) Applicant: Chad & Barneys HPF, Inc., Liberty Lake, WA (US)

(72) Inventors: Rhett V. Barney, Liberty Lake, WA (US); Chad T. Robinson, Folsom, CA (US)

(21) Appl. No.: 19/169,944

(22) Filed: Apr. 3, 2025

(51) Int. Cl. A23L 23/00; A23J 3/04; A23J 3/08; A61K 36/41

(52) U.S. Cl. CPC A23L 23/00; A23J 3/04; A23J 3/08; A61K 36/41

(57) ABSTRACT

An adaptogenic, high-protein (AHP) food product, such as a sauce, and methods of producing the AHP food product are disclosed herein. The AHP food product is produced by combining, in any order, a collagen peptide, a whey protein, a bone broth protein, adaptogens, and a food base. The AHP food product can further include an edible acid and/or additional additives.

### FIELD OF THE DISCLOSURE

The current disclosure describes an adaptogenic, high-protein (AHP) food product, such as a sauce. The present disclosure also provides a method of producing the AHP food product. The present disclosure uses adaptogens to provide adaptogenic, anti-inflammatory, and metabolic support to a high protein food product. In particular embodiments, the AHP food product is produced by combining, in any order, a combination of proteins and adaptogens best suited for the food base it is being placed into. In particular embodiments, the combination of proteins includes collagen peptide, a whey protein, and a bone broth protein. In further embodiments, the AHP food product further includes an edible acid. In particular embodiments, the adaptogens include a mushroom blend, holy basil (Tulsi), ashwaganda, Rhodiola Rosea, and/or turmeric (curcumin). In particular embodiments, the mushroom blend includes reishi, Lion's Mane, cordyceps, chaga, and/or Turkey tail. In further embodiments, the AHP food product further includes additional additives.

### BACKGROUND OF THE DISCLOSURE

In the body, proteins are a part of every cell and tissue, including muscle. Dietary protein is required to promote growth, repair damaged cells and tissue, synthesize hormones, and are important in a variety of metabolic activities. Proteins contain essential and non-essential amino acids. Amino acids are the building blocks for all our proteins in the body. Essential amino acids are those that the human body cannot make on its own and thus, these amino acids must come from eating foods containing those amino acids. There are multiple sources of proteins available; however, animal sources of protein often contain all of the essential amino acids and are considered complete sources of protein, whereas most plant proteins lack some of the essential amino acids and are therefore classified as incomplete.

The daily recommended allowance (RDA) for protein can be calculated by a person's body weight. For example, the Academy of Nutrition and Dietetics recommends that the average individual should consume 0.8 grams of protein per kilogram or 0.35 grams per pound of body weight per day for general health. This equates to 60 g of protein per day for a 75 kg (165 pound) person. Furthermore, research shows that protein intake should increase with age and increase for individuals with active lifestyles. Around age 50, one gram of protein per kg of body weight is recommended to maintain muscle mass. The American College of Sports Medicine advocates a protein intake of between 1.2-1.4 g per kg body weight per day for individual partaking in endurance sports and 1.7 g per kg body weight per day for individuals participating in power disciplines such as strength or speed training. Similar conclusions were drawn by the International Society of Sports Nutrition, even suggesting ranges up to 2.0 g per kg body weight per day (150 g protein per day for a 75 kg person) for people with active lifestyles. For people aiming to hit high levels of protein per day to gain muscle mass, reaching protein goals while balancing fat, carbohydrates, and calorie intake can be difficult. This difficulty comes in part because of the volume required, as well as the lack of variety of available high-quality high protein foods.

### SUMMARY OF THE DISCLOSURE

The present disclosure describes an adaptogenic, high-protein (AHP) food product (e.g., AHP sauce) and a method of making the AHP food product. The present disclosure uses adaptogens to provide adaptogenic, anti-inflammatory, and metabolic support to a high protein food product. In particular embodiments, the AHP food product is prepared by combining, in any order, a collagen peptide, a whey protein, a bone broth protein, adaptogens, and a food base. In further embodiments, the pH of the AHP food product is adjusted by admixing an edible acid. A combination of protein sources are included in order to balance the benefits and drawbacks of each protein source such that the final food product has a high protein content that is complete, has an appealing texture, and an appetizing taste with the added benefits of the adaptogens.

Adaptogens provide the AHP food product with anti-inflammatory, metabolic, and immune support. In particular embodiments, the adaptogens include a mushroom blend, holy basil (Tulsi), ashwaganda, Rhodiola Rosea, and/or turmeric (curcumin). In particular embodiments, the mushroom blend includes reishi, Lion's Mane, cordyceps, chaga, and/or Turkey tail. In particular embodiments, the food product includes adaptogens at a concentration by weight of 0.01-7%. In particular embodiments, the food product includes adaptogens at a concentration by weight of 0.03-6%. In particular embodiments, the food product includes adaptogens at a concentration by weight of 0.05-5%.

Collagen peptides are becoming a popular protein source because of the proposed benefits of improved skin health and reduced joint pain. The popularity of collagen peptides as a supplement has also increased because of the ease of which it can be added to everyday foods and beverages due to its ability to dissolve in hot or cold water. Collagen, however, is not a complete protein and lacks the amino acid tryptophan. Furthermore, it is generally believed that proteins high in branched chain amino acids (BCAA), such as leucine, are important when considering a protein supplement. Collagen itself is considered to have low amounts of BCAA and lysine. As such, additional protein sources to supplement the addition of collagen peptides to produce the AHP food product are desired.

The dairy-based protein, whey protein, is beneficial in supporting muscle adaptations due to its rapid absorption rate. Whey protein is one of the most common protein supplements because of its many benefits including being considered a complete protein, containing nine essential amino acids. Unfortunately, like most dairy products, whey proteins can coagulate when denatured. In other words, when heat or acid are added to whey proteins, the proteins denature, exposing “sticky” portions of their structure that aggregate, forming lumps. In preparing many food products, inclusion of too much whey protein can cause the food product to appear clumpy and curdled during cooking or during packaging (e.g., during a hot-fill process). To prepare an AHP food product, a balance between the protein content and avoidance of coagulation must be reached.

Bone broth protein is another excellent source of crucial amino acids and protein. In addition to its protein content, bone broth is considered to be very nutritious as it contains several vitamins and minerals. Depending on the bone broth protein, like whey protein, it can also be considered a complete protein. Despite the nutritious benefits of bone broth protein, it can have strong flavors and can skew the taste profile of food products to which it is added. To prepare an AHP food product, it is critical that the added protein content from bone broth protein and its effect on the flavor are balanced.

In particular embodiments, the AHP food product includes a protein mixture, adaptogens, and a food base. In particular embodiments, the protein mixture includes a collagen peptide, a whey protein, and/or a bone broth protein. In particular embodiments, the food product includes a protein mixture at a concentration by weight of 1-25%. In particular embodiments, the food product includes a protein mixture at a concentration by weight of 5-20%. In particular embodiments, the food product includes a protein mixture at a concentration by weight of 10-18%.

The multiple protein sources can be added to a food base to increase the protein content of the food base thereby producing an AHP food product. In particular embodiments, an AHP food product includes a food base. The food base is the comestible to which the protein mixture (e.g., collagen peptide, whey protein, and bone broth protein) and adaptogens are added. In particular embodiments, the food base is a sauce including a pasta sauce, a condiment, taco sauce, barbecue sauce, soup base, chili sauce, hot sauce, curry, masala, chutney, or a salsa. In particular embodiments, the food base includes a pasta sauce. In particular embodiments, the AHP food product includes a food base at a concentration by weight of 50-99%. In particular embodiments, the AHP food product includes a food base at a concentration by weight of 70-90%. In particular embodiments, the AHP food product includes a food base at a concentration by weight of 75-85%.

In order to make the AHP food product shelf stable, its pH can be adjusted to an acidic pH. In particular embodiments, the acidic pH includes 3.0-4.8. One method of controlling the pH is to add an edible acid. In particular embodiments, an edible acid includes citric acid or vinegar. The addition of the protein mixture to the food base often increases the pH of the resulting food product and the edible acid can lower the pH back to desired levels. In some cases, the pH of the food base will change the amount of edible acid needed to reach a desired pH level. The higher the pH, the more edible acid needed to reach the desired pH level. In addition, the pH of the AHP food product can affect the thermal stability of the proteins (e.g., whey) within the AHP food product. For example, whey protein has been found to coagulate when heated to 95° C. at a pH above 3.8 but depending on the amount of whey protein, the same heat can be applied at a pH below 3.7 without precipitation of the protein (Bernal V and P Jelen, Journal of Dairy Science 68 (11): 2847-2852, 1985). Unfortunately, edible acids can also change the taste of the resulting food product and a balance must be reached to maintain pH and taste. In particular embodiments, the AHP food product includes an edible acid at a concentration by weight of 0-2%. In particular embodiments, the AHP food product includes an edible acid at a concentration by weight of 0.3-1.5%. In particular embodiments, the AHP food product includes an edible acid at a concentration by weight of 1.2%.

The present disclosure provides a AHP food product (e.g., AHP sauce) made of a delicate balance of collagen peptides, whey protein, bone broth protein, adaptogens, food base, and edible acid to enhance the protein content of the AHP food product, providing high protein content and adaptogens, while maintaining taste and texture of the resulting food product.

With its staged cooking process, the potency of adaptogenic and anti-inflammatory ingredients is maximized in an acidic food base that stabilizes and enhances bioavailability. This formulation delivers multi-functional support, including stress relief, immune resilience, metabolic boost, and cognitive support, all in a delicious, balanced food product (e.g., sauce).

### DETAILED DESCRIPTION

The present disclosure describes a AHP food product (e.g., AHP sauce) and method of making the AHP food product. In particular embodiments, the AHP food product (or food product) is prepared by combining, in any order, a collagen peptide, a whey protein, a bone broth protein, adaptogens, and a food base. In further embodiments, the pH of the food product is adjusted by admixing an edible acid. A combination of protein sources are included in order to balance the benefits and drawbacks of each protein source such that the final food product has a high protein content, has an appealing texture, and an appetizing taste with the added benefits of adaptogens. In particular embodiments, the food product includes additional additives. In particular embodiments, additional additives include vitamins, minerals, flavoring agents, sweeteners, taste-masking agents (e.g., bitter blockers), colorants, or food-grade preservatives.

Adaptogens provide the AHP food product with anti-inflammatory, metabolic, and immune support. In particular embodiments, the adaptogens include a mushroom blend, holy basil (Tulsi), ashwaganda, Rhodiola Rosea, and/or turmeric (curcumin). In particular embodiments, the mushroom blend includes reishi, Lion's Mane, cordyceps, chaga, and/or Turkey tail. In particular embodiments, the food product includes adaptogens at a concentration by weight of 0.01-7% of the food product. In particular embodiments, the food product includes adaptogens at a concentration by weight of 0.03-6% of the food product. In particular embodiments, the food product includes adaptogens at a concentration by weight of 0.05-5% of the food product. In particular embodiments, the adaptogens include Rhodiola Rosea. In particular embodiments, the food product includes Rhodiola Rosea at a concentration by weight of 0.01-7% of the food product. In particular embodiments, the food product includes Rhodiola Rosea at a concentration by weight of 0.03-6% of the food product. In particular embodiments, the food product includes Rhodiola Rosea at a concentration by weight of 0.05-5% of the food product.

Collagen peptides are becoming a popular protein source because of the proposed benefits of improved skin health and reduced joint pain. The popularity of collagen peptides as a supplement has also increased because of the ease of which it can be added to everyday foods and beverages due to its ability to dissolve in hot or cold water. Collagen, however, is not a complete protein and lacks the amino acid tryptophan. Furthermore, it is generally believed that proteins high in branched chain amino acids (BCAA), such as leucine, are important when considering a protein supplement. Collagen itself is considered to have low amounts of BCAA and lysine. As such, additional protein sources to supplement the addition of collagen peptides to produce the food product are desired.

The dairy-based protein, whey protein, is beneficial in supporting muscle adaptations due to its rapid absorption rate. Whey protein is one of the most common protein supplements because of its many benefits including being considered a complete protein, containing nine essential amino acids. Unfortunately, like most dairy products, whey proteins can coagulate when denatured. In other words, when heat and sometimes acid are added to whey proteins, the proteins denature, exposing “sticky” portions of their structure that aggregate, forming lumps. In preparing many food products, inclusion of too much whey protein can cause the food product to appear clumpy and curdled during cooking or during packaging (e.g., during a hot-fill process). To prepare a food product, a balance between the protein content and avoidance of coagulation must be reached.

Bone broth protein is another excellent source of crucial amino acids and protein. In addition to its protein content, bone broth is considered to be very nutritious as it contains several vitamins and minerals. Depending on the bone broth protein, like whey protein, it can also be considered a complete protein. Despite the nutritious benefits of bone broth protein, it can have strong flavors and can skew the taste profile of food products to which it is added. To prepare a food product, it is critical that the added protein content from bone broth protein and its effect on the flavor are balanced.

In particular embodiments, the food product includes a protein mixture (e.g., collagen peptide, whey protein, and bone broth protein) at a concentration by weight of 1-25%. In particular embodiments, the food product includes a protein mixture at a concentration by weight of 5-20%. In particular embodiments, the food product includes a protein mixture at a concentration by weight of 10-18%.

Multiple protein sources can be added to a food base to increase the protein content of the food base thereby producing a food product. In particular embodiments, a food product includes a food base. The food base is the comestible to which the protein mixture (e.g., collagen peptide, whey protein, and bone broth protein) is added. In particular embodiments, the food base is a sauce including a pasta sauce, a condiment, taco sauce, barbecue sauce, soup base, chili sauce, hot sauce, curry, masala, chutney, or a salsa. In particular embodiments, the food base includes a pasta sauce.

In order to make the food product shelf stable, its pH can be adjusted to an acidic pH. A “shelf stable” food product is a food product that can be stored at room temperature. In particular embodiments, the acidic pH includes 3.0-4.8. One method of controlling the pH is to add an edible acid. In particular embodiments, an edible acid includes citric acid. In particular embodiments, an edible acid includes vinegar. The addition of the protein mixture to the food base often increases the pH of the resulting food product and the edible acid can lower the pH back to desired levels. In particular embodiments, the pH of either the food base, the adaptogens, or protein mixture can vary from batch to batch, thus the amount of edible acid needed to reach the desired pH level may vary.

The higher the pH, the more edible acid needed to reach the desired pH level. Unfortunately, edible acids can also change the taste of the resulting food product and a balance must be reached to maintain pH and taste.

The present disclosure provides a food product made of a delicate balance of collagen peptides, whey protein, bone broth protein, adaptogens, food base, and edible acid to enhance the protein content of the food product while maintaining taste and texture of the resulting food product. Maintaining taste means that the flavor and taste of the food product does not significantly vary from the flavor and taste of the food base. In particular embodiments, taste can be maintained using additional additives such as taste-masking agents (e.g., bitter blockers). Herein, “does not significantly vary” means that persons of average perception of taste rate the flavor to be similar. In particular embodiments, the food product includes at least 1 weight %, 2 weight %, at least 3 weight %, at least 5 weight % protein. In particular embodiments, the food product includes at least 0.01 weight %, at least 0.02 weight %, at least 0.03 weight %, at least 0.04 weight %, at least 0.05 weight %, at least 0.07 weight %, at least 0.1 weight %, at least 0.3 weight %, at least 0.5 weight %, at least 0.7 weight %, at least 1 weight %, at least 2 weight %, at least 3 weight %, or at least 4 weight % adaptogens.

The food product refers to the final composition. In particular embodiments, the food product contains the food base, adaptogens, collagen peptide, whey protein, and bone broth protein.

In particular embodiments, the food product includes a protein mixture at a concentration by weight of 1-25% of the food product, adaptogens at a concentration by weight of 0.01-7% of the food product, and a food base at a concentration by weight of 50-99% of the food product, wherein the selected amounts do not exceed 100%. In particular embodiments, the food product includes a protein mixture at a concentration by weight of 1-25% of the food product, adaptogens at a concentration by weight of 0.01-7% of the food product, a food base at a concentration by weight of 50-99% of the food product, and an edible acid at a concentration by weight of 0-2% of the food product, wherein the selected amounts do not exceed 100%. In particular embodiments, the protein mixture includes collagen peptide, whey protein, and/or bone broth protein. In particular embodiments, the protein mixture includes additional or alternative proteins. In particular embodiments, additional or alternative proteins include casein protein, soy protein, egg protein, pea protein, wheat protein, hemp protein, or rice protein.

In particular embodiments, the food product includes a protein mixture at a concentration by weight of 5-20% of the food product, adaptogens at a concentration by weight of 0.03-6% of the food product, and a food base at a concentration by weight of 70-90% of the food product, wherein the selected amounts do not exceed 100%. In particular embodiments, the food product includes a protein mixture at a concentration by weight of 5-20% of the food product, adaptogens at a concentration by weight of 0.03-6% of the food product, and a food base at a concentration by weight of 70-90% of the food product, and an edible acid at a concentration by weight of 0.3-1.5% of the food product, wherein the selected amounts do not exceed 100%.

In particular embodiments, the food product includes a protein mixture at a concentration by weight of 10-18% of the food product, adaptogens at a concentration by weight of 0.05-5% of the food product, and a food base at a concentration by weight of 75-85% of the food product, wherein the selected amounts do not exceed 100%. In particular embodiments, the food product includes a protein mixture at a concentration by weight of 10-18% of the food product, adaptogens at a concentration by weight of 0.05-5% of the food product, and a food base at a concentration by weight of 75-85% of the food product, and an edible acid at a concentration by weight of 1.2% of the food product, wherein the selected amounts do not exceed 100%.

In particular embodiments, alternative or additional proteins can be used within the protein mixture. In particular embodiments, the food product includes additional additives. In particular embodiments, additional additives include vitamins, minerals, flavoring agents, sweeteners, taste-masking agents (e.g., bitter blockers), colorants, or food-grade preservatives. In particular embodiments, the food product includes additional additives at a concentration by weight of less than 1%, less than 0.1%, less than 0.01%, or less than 0.001%. In particular embodiments, the food product includes taste-masking agents.

Developing an AHP food product uses a staged cooking process to maintain ingredient stability. The process disclosed herein allows for precise heat management to ensure that each adaptogen is added at an optimal stage during the cooking process to preserve its unique properties. This ensure maximum potency and effectiveness.

The AHP food product includes combinations of synergistic ingredients in an acidic environment, natural flavor masking and balancing, and synergistic anti-inflammatory and immune-boosting properties. For example, the combination of five mushrooms, adaptogens like holy basil, turmeric, and rhodiola, in a tomato-based sauce provides synergistic health benefits while the acidic environment enhances stability and bioavailability, making this formula both unique and effective. In particular embodiments, using a tomato's natural acidity and umami, along with a touch of natural sweeteners, a balanced flavor profile can mask the bitter flavors of adaptogens. Furthermore, the combination of lycopene with adaptogenic and anti-inflammatory ingredients like turmeric, holy basil, and/or chaga creates a comprehensive anti-inflammatory and immune-support profile, making the food product beneficial for holistic wellness.

Aspects of the current disclosure are now described in more supporting detail as follows: (i) Protein Mixture; (i-a) Collagen Peptide; (i-b) Whey Protein; (i-c) Bone Broth Protein; (i-d) Additional Protein Sources; (ii) Adaptogens; (iii) Probiotics; (iv) Fiber Mixture; (v) Food Base; (vi) Edible Acid; (vii) Composition of Food Product; (viii) Preparation of the Composition; (ix) Exemplary Embodiments; (x) Examples; and (xi) Closing Paragraphs. These headings are provided for organizational purposes only and do not limit the scope or interpretation of the disclosure.

(i) Protein Mixture. In the context of this disclosure, a “protein mixture” refers to a collection of proteins, proteinaceous matter, peptides and amino acids, free or in any bound form, that is added to the food base to elevate the protein content of the resulting food product. The term “elevated protein content” refers to an increase in protein content compared to a reference level. In particular embodiments, the reference level is an amount of protein content in a food product without added protein. In particular embodiments, the reference level is an amount of protein content in previously available food products. In particular embodiments, the reference level is the protein content of a food base used to make the food product.

In particular embodiments, the protein mixture used for compositions of food products include any combination of proteins. In particular embodiments, the protein mixture used for compositions of food products disclosed herein include a collagen peptide, a whey protein, and a bone broth protein. The protein mixture can include hydrolyzed proteins and/or non-hydrolyzed proteins. The protein mixture can include protein concentrates, protein isolates, or protein hydrolysates. Protein concentrates are produced by extracting protein from whole food using heat and acid or enzymes. Protein concentrates typically supply 60%-80% protein, with the remaining 20%-40% composed of fat and carbs. Protein isolates are made through an additional filtering process, which removes more fat and carbs, further concentrating the protein. Protein isolate powders typically contain 90%-95% protein. Protein hydrolysates are produced by further heating with acid or enzymes, which breaks the bonds between amino acids. Protein hydrolysates are absorbed more quickly by your body and muscles.

A protein can be defined by its quality or grade. In particular embodiments, protein grade is calculated from lowest grade to highest grade as C, C+, B, B+, A, A+, or A++, wherein A++. Protein quality or protein grade is defined by a number of methods. The protein digestibility-corrected amino acid score (PDCAAS) is a method of evaluating the quality of a protein according to the amino acid requirements of a human and its digestibility. The protein efficiency ratio (PER) is a method of evaluating the quality of a protein based on the weight gain of a subject divided by its intake of a particular food protein during a period of time. A protein ratio can also be determined by dividing the serving size from the amount of protein in one serving. In addition to protein ratio, other considerations determining protein quality include the protein source, bioavailability of the protein, amino acid profile, taste, and ability to dissolve. In particular embodiments, a higher-grade protein (e.g., A, A+, or A++) dissolves better than lower-grade protein. In particular embodiments, a higher-grade protein has a better taste profile (e.g., less bitter) compared to lower-grade protein. In particular embodiments, the protein mixture has a protein grade of C, C+, B, B+, A, A+, or A++. In particular embodiments, the protein mixture has a protein grade of A, A+, or A++. In particular embodiments, the collagen peptide, whey protein, and/or bone broth protein each have a protein grade of C, C+, B, B+, A, A+, or A++. In particular embodiments, the collagen peptide has a protein grade of A, A+, or A++. In particular embodiments, the whey protein has a protein grade of A, A+, or A++. In particular embodiments, the bone broth protein has a protein grade of A, A+, or A++.

In particular embodiments, the protein mixture includes hydrolyzed protein and/or non-hydrolyzed protein. In particular embodiments, the protein mixture includes hydrolyzed protein. A “hydrolyzed protein” is a mixture of amino acids, peptides, and polypeptides created by breaking down proteins through a process called hydrolysis. A hydrolyzed protein is a protein that has undergone hydrolysis. Hydrolysis is a process in which peptide bonds in the protein's structure are broken down into smaller fragments, such as amino acids or peptides. Hydrolysis may be achieved through chemical methods or enzymatic methods. A non-hydrolyzed protein is a protein that has not undergone hydrolysis. A non-hydrolyzed protein can be in its intact, native form with long chains of amino acids linked by peptide bonds. Non-hydrolyzed proteins are less broken down than hydrolyzed proteins and may require further digestion during metabolic processes.

In particular embodiments, the protein mixture includes one or more hydrolyzed proteins in an amount of 1 wt %, 2 wt %, 3, wt %, 4, wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, 50 wt %, or more by weight of the protein mixture. In particular embodiments, the ratio of hydrolyzed protein to non-hydrolyzed protein includes 1:0, 99:1, 49:1, 97:3, 24:1, 19:1, 47:3, 93:7, 23:2, 91:9, 9:1, 89:11, 88:12, 87:13, 43:7, 85:15, 21:4, 83:17, 82:18, 81:19, 4:1, 79:21, 39:11, 77:23, 76:24, 3:1, 37:13, 73:27, 36:14, 71:29, 70:30, 69:31, 34:16, 67:33, 66:34, 65:35, 32:18, 63:37, 31:19, 61:39, or 3:2.

Hydrolysis of the protein can increase the solubility of the protein. Soluble protein is protein that does not precipitate (i.e., resides in the supernatant) when a 2% (w/w) suspension of the total protein in water is centrifuged at 31,000×g at 20 degrees Celsius for sixty minutes.

A hydrolyzed protein has favorable characteristics due to the increased degree of hydrolysis and solubility such as: emulsion capacity, digestibility, antioxidant activity, and mineral delivery. The food product herein balances these benefits with the taste and texture of the resulting food product.

(i-a) Collagen Peptide. The term collagen peptide refers to a supplement containing short chains (i.e., peptides) extracted from native (full-length) animal collagen. The collagen peptide can include additional or filler ingredients such as thickeners, flavor enhancers, gums, preservatives, etc. The collagen peptide used in the present disclosure may be obtained by subjecting a modified collagen such as collagen or gelatin to hydrolysis treatment with an enzyme, an acid, an alkali or the like, or may be an artificially synthesized collagen peptide, or 1 or 2 or more of these. Examples of the collagen and gelatin include collagen and gelatin derived from animals such as cattle, pigs, and chickens, collagen and gelatin derived from fish. In particular embodiments, the collagen peptide is bovine-derived.

Collagen can be obtained from the bones, connective tissue, skin parts, fish bones, skins, scale parts, etc. of the animal, and various known materials such as bones may be subjected to conventionally known treatments such as degreasing treatment and extraction treatment. In order to remove dirt and contaminants attached to the materials (such as for fish scales), a washing process such as washing with water is performed several times in advance to remove the dirt and contaminants, and degreasing treatment is performed to remove fats and oils. In particular embodiments, inorganic substances such as phosphorus and calcium are removed by ash treatment.

The enzyme for producing the collagen peptide may be any enzyme capable of cleaving the peptide bond of collagen or gelatin, and examples thereof include collagenase, papain, bromelain, actinidin, ficin, cathepsin, pepsin, rennin, trypsin, and enzyme preparations containing these enzymes. As an acid, for example, hydrochloric acid, sulfuric acid, nitric acid, or the like can be used. As a base, for example, sodium hydroxide, calcium hydroxide, or the like can be used.

In the present disclosure, the collagen peptide aqueous solution may be used as it is, or may be used after being powdered by drying or the like. The aqueous solution may be subjected to a purification treatment usually used, and the purified aqueous solution may be used in the form of an aqueous solution, powder, or the like. In particular embodiments, the collagen peptide is in the form of a powder.

In addition, collagen peptides of all molecular weights, such as collagen peptides having a molecular weight of 10,000 or less; 100 to 10,000; 100 to 7,000; or 100 to 5,000 can be used. The molecular weight of the collagen peptide can be measured by a known quantitative method such as high-performance liquid chromatography (HPLC) and gel filtration. In general, collagen peptides have a lower in vivo absorbability as the molecular weight thereof is larger. Alternatively, collagen peptides having a lower molecular weight have undesirable tastes (bitterness, astringency, etc.).

The collagen peptide may be an extract and/or a purified product. In particular embodiments, the collagen peptide includes a purity of 50 weight % or more, 70 weight % or more, or 90 weight % or more. In particular embodiments, the collagen peptide can be a commercially available product, for example, “collagen peptide SCP-5100” (manufactured by neotame gelatin Co., Ltd.).

In particular embodiments, the collagen peptide includes at least 0.5 g, at least 0.6 g, at least 0.7 g, at least 0.8 g, or at least 0.9 g of protein per gram of collagen peptide. In particular embodiments, the collagen peptide includes at least 0.8 g, at least 0.81 g, at least 0.82 g, at least 0.83 g, at least 0.84 g, at least 0.85 g, at least 0.86 g, at least 0.87 g, at least 0.88 g, at least 0.89 g, at least 0.9 g, at least 0.91 g, at least 0.92 g, at least 0.93 g, at least 0.94 g, or at least 0.95 g of protein per gram of collagen peptide. In particular embodiments, the collagen peptide includes 0.81 g of protein per gram of collagen peptide. In particular embodiments, the collagen peptide does not include fat.

In particular embodiments, the food product includes a collagen peptide at a concentration by weight of 0-20%. In particular embodiments, the food product includes a collagen peptide at a concentration by weight of 0-18%. In particular embodiments, food product includes a collagen peptide at a concentration by weight of 0-17%. In particular embodiments, the food product includes a collagen peptide at a concentration by weight of at least 1%, at least 5%, at least 6%, at least 7%, at least 8%, at least 9%, at least 10%, at least 11%, at least 12%, at least 13%, at least 14%, at least 15%, at least 16%, or at least 20%.

(i-b) Whey Protein. One of the most commonly used classes of food protein is whey protein. It is known for its excellent amino acid profile, for its ability to increase the protein synthesis in a mammal (due to a higher leucine content), for its improved tolerance and increased gastric emptying, and for its interesting bioactive proteins with immune enhancing properties (lactoglobulins, immunoglobulins, lysozyme, glutamine, cysteine and lactoferrins). Nutritionally speaking, whey protein is known as a naturally complete protein because it contains all of the essential amino acids required in the daily diet. It is also one of the richest sources of branched chain amino acids (BCAAs, in particular leucine) which play an important role in muscle protein synthesis. Moreover, some of the individual components of whey protein have been shown to prevent viral and bacterial infection and modulate immunity in animals. Whey protein is the preferred choice of proteins to treat persons suffering from sarcopenia, but is also suitable for healthy persons, such as sportsmen and active elderly.

Herein, whey protein refers to any available protein isolated from whey, the liquid portion of milk that separates from the curds when making cheese. Whey protein includes whey protein isolate and whey protein concentrate. The whey protein can be in the form of a dry powder or in a liquid form. The whey protein can include additional or filler ingredients such as thickeners, flavor enhancers, gums, preservatives, etc.

As a source of whey protein to be used in the present disclosure, any commercially available whey protein source may be used, i.e. whey obtained by any process for the preparation of whey known in the art, as well as whey protein fractions prepared thereof, or the proteins that constitute the bulk of the whey proteins being β-lactoglobulin, α-lactalbumin and serum albumin, such as liquid whey, or whey in powder form, such as whey protein isolate (WPI) or whey protein concentrate (WPC). Whey protein concentrate is rich in whey proteins, but also contains other components such as fat, lactose and glycomacroprotein (GMP), a casein-related non-globular protein. Typically, whey protein concentrate is produced by membrane filtration. On the other hand, whey protein isolate includes primarily whey proteins with minimal amounts of fat and lactose. Whey protein isolate usually requires a more rigorous separation process such as a combination of microfiltration and ultra-filtration or ion exchange chromatography. It is generally understood that a whey protein isolate refers to a mixture in which at least 90 weight % of the solids are whey proteins. A whey protein concentrate is understood as having a percentage of whey proteins between the initial amount in the by-product (12 weight %) and a whey protein isolate. In particular, sweet whey, obtained as a by-product in the manufacturing of cheese, acid whey, obtained as a by-product in the manufacturing of acid casein, native whey, obtained by milk microfiltration or rennet whey, obtained as a by-product in the manufacturing of rennet casein, may be used alone or in combination as source of globular whey proteins. In particular embodiments, the whey protein is a whey protein isolate.

Furthermore, whey proteins may originate from all kinds of mammalian animal species, such as, for instance cows, sheep, goats, horses, buffaloes, and camels. In particular embodiments, the whey protein is of bovine origin.

In particular embodiments, the whey protein is available as a powder. In particular embodiments, the whey protein includes at least 0.1 g, at least 0.3 g, at least 0.5 g, at least 0.6 g, at least 0.7 g, at least 0.8 g, or at least 0.9 g of protein per gram of whey protein. In particular embodiments, the whey protein includes at least 0.8 g, at least 0.81 g, at least 0.82 g, at least 0.83 g, at least 0.84 g, at least 0.85 g, at least 0.86 g, at least 0.87 g, at least 0.88 g, at least 0.89 g, at least 0.9 g, at least 0.91 g, at least 0.92 g, at least 0.93 g, at least 0.94 g, or at least 0.95 g of protein per gram of whey protein. In particular embodiments, the whey protein includes 0.86 g of protein per gram of whey protein. In particular embodiments, the whey protein does not include fat.

In particular embodiments, the food product includes a whey protein at a concentration by weight of 0-4%. In particular embodiments, the food product includes a whey protein at a concentration by weight of 0-2%. In particular embodiments, the food product includes a whey protein at a concentration by weight of 0-0.7%. In particular embodiments, the food product includes a whey protein at a concentration by weight of at least 0.1%, at least 0.2%, at least 0.3%, at least 0.4%, at least 0.5%, at least 0.6%, at least 0.7%, at least 0.8%, at least 1.0%, at least 1.3%, at least 1.5%, at least 1.7%, at least 1.9%, or at 2.0%. In particular embodiments, the food product includes a whey protein at a concentration by weight of at least 0.44%, at least 0.45%, at least 0.46%, at least 0.47%, at least 0.48%, at least 0.49%, at least 0.5%, at least 0.61%, at least 0.62%, at least 0.63%, at least 0.64%, at least 0.65%, at least 0.66%, at least 0.67%, at least 0.68%, or at least 0.69%.

(i-c) Bone Broth Protein. Bone broth protein refers to any supplement or protein derived from bone broth or the cooking of animal bones and connective tissues. The bone broth protein can be in a liquid form or in the form of a powder. The bone broth protein includes protein concentrates, protein isolates, or protein hydrolysates. The bone broth protein can include additional or filler ingredients such as thickeners, flavor enhancers, gums, preservatives, etc.

Bone broth is a nutrient-filled stock made from slowly simmered animal bones and connective tissues. There are many different types of bone broth which can be derived from several kinds of animal species including chicken, cows, fish, turkey, lamb, goat, quail, and many kinds of large and small game mammals and birds. In particular embodiments, bone broth includes collagen protein, a range of important amino acids, minerals, and other compounds including glucosamine, chondroitin, and hyaluronic acid. Amino acids are important in several biological processes and aid in normal muscle growth, hormone production, healthy cognitive function, appetite control and more. In particular embodiments, bone broth includes type II collagen. Type II collagen is the type of collagen found in the lining of the gut, joints, and cartilage. It is important in building and promoting healthy connective tissues. Glucosamine chondroitin, and hyaluronic acid are important compounds for supporting joints and cartilage. These compounds allow joints to move smoothly and absorb shock. Furthermore, these compounds are less common in protein sources.

Bone broth protein can vary by composition and particle size based on the starting materials and process of manufacture. In general, bone broth protein contains animal protein including collagen; peptides and amino acids derived from collagen; glycosaminoglycans including glucosamine, chondroitin and hyaluronic acid; carbohydrates; sodium; potassium; and calcium. In particular embodiments, the bone broth protein includes at least 60%, at least 65%, at least 70%, at least 71%, at least 72%, at least 73%, at least 74%, at least 75%, at least 76%, at least 77%, at least 78%, at least 79%, or at least 80% protein. In particular embodiments, the bone broth protein includes less than 25%, less than 20%, less than 15%, %, less than 14%, less than 13%, less than 12%, less than 11%, less than 10%, less than 9%, less than 8%, less than 7%, less than 6%, or less than 5% carbohydrates. In particular embodiments, the bone broth protein includes less than 15%, less than 10%, less than 9%, less than 8%, less than 7%, less than 6%, less than 5%, less than 4%, less than 3%, less than 2%, less than 1%, less than 0.7%, or less than 0.5% sodium. In particular embodiments, the bone broth protein includes 0.1 weight %, 0.3 weight %, 0.5 weight %, 0.7 weight %, 0.8 weight %, 0.9 weight %, 1 weight %, 1.1 weight %, 1.2 weight %, 1.3 weight %, 1.5 weight %, 1.8 weight %, 2 weight %, or 3 weight % potassium. In particular embodiments, the bone broth protein includes 0.01 weight %, 0.02 weight %, 0.03 weight %, 0.04 weight %, 0.05 weight %, 0.06 weight %, 0.07 weight %, 0.08 weight %, 0.09 weight %, 0.1 weight %, 0.2 weight %, 0.3 weight % calcium. In particular embodiments, the bone broth protein does not include any discernible dietary fiber content. In particular embodiments, the bone broth protein has a fat content of less than 4 weight %, less than 3.5 weight %, less than 3.0 weight %, less than 2.5 weight %, less than 2.0 weight %, less than 1.5 weight %, less than 1.0 weight %, less than 0.5 weight %, or less than 0.1 weight %. In particular embodiments, the bone broth protein has a fat content of 0 g.

Bone broth protein can be prepared by a number of methods known to one of skill in the art. Typically bone broth protein is prepared by cooking bones and connective tissues in aqueous media (e.g., water) thereby preparing an aqueous extract, concentrating the aqueous extract, and drying and agglomerating the concentrated aqueous extract.

In particular embodiments, the cooking includes applying a temperature of 185° F.-230° F., 190° F.-215° F., or 200° F.-212° F. for a select cooking time. In particular embodiments, the select cooking time includes at least 10 min, at least 20 min, at least 30 min, at least 40 min, at least 50 min, or at least an hour. In particular embodiments, the cooking can include applying temperature at an elevated pressure. In particular embodiments, the aqueous extract includes aqueous media (e.g., water) and at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, or at least 30% by weight associated with bones and connective tissues. In particular embodiments, the method further includes removing fat by skimming the surface or gravity separation. Often, fat and other undesirable components float to the top of the aqueous extract and can be removed by skimming the top. In particular embodiments, concentrating the aqueous extract includes concentrating by evaporating, concentration by reverse osmosis, concentration by direct osmosis, concentration by membrane distillation, concentration by osmotic distillation, or freeze concentration. In particular embodiments, drying includes spray drying. Agglomeration technology has been well known for decades and is well within the skill of the art. Agglomeration is what helps to make the powders instant, meaning that the user may consume the powder as is or partially or completely reconstituted in one or more liquids but without necessity of further heating, cooking or other preparation. In particular embodiments, aqueous extract is filtered through a 10 μm-3 mm screen. In particular embodiments, aqueous extract is filtered through a 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, or 1000 μm screen. In particular embodiments, the resulting powder is sifted through a 100 μm-5000 μm screen. In particular embodiments, the resulting powder is sifted through a 500 μm, 750 μm, 1000 μm, 1250 μm, 1500 μm, 1750 μm, 2000 μm, 2250 μm, 2500 μm, or 3000 μm screen.

In some protocols, the bone broth protein is prepared by cooking bones and connective tissues in water at 212° F. for at least 30 minutes to prepare an aqueous extract including 25% by weight associated with bones and connective tissues. While cooking, the fat fraction is removed from the surface by skimming or gravity separation. The aqueous extract is then filtered through a 300 μm screen, spray dried and agglomerated, and the resulting powder is sifted through a 2000 μm screen. After passing the resulting powder through the field of a rare earth magnet, the powder is packed in hermetically sealed plastic bag packaging.

In particular embodiments, the bone broth protein includes at least 0.1 g, at least 0.3 g, at least 0.5 g, at least 0.6 g, at least 0.7 g, at least 0.8 g, or at least 0.9 g of protein per gram of bone broth protein. In particular embodiments, the bone broth protein includes at least 0.8 g, at least 0.81 g, at least 0.82 g, at least 0.83 g, at least 0.84 g, at least 0.85 g, at least 0.86 g, at least 0.87 g, at least 0.88 g, at least 0.89 g, at least 0.9 g, at least 0.91 g, at least 0.92 g, at least 0.93 g, at least 0.94 g, or at least 0.95 g of protein per gram of bone broth protein. In particular embodiments, the bone broth protein includes 0.81 g of protein per gram of bone broth protein. In particular embodiments, the bone broth protein does not include fat.

In particular embodiments, the food product includes a bone broth protein at a concentration by weight of 0-10%. In particular embodiments, the food product includes a bone broth protein at a concentration by weight of 0-5%. In particular embodiments, the food product includes a bone broth protein at a concentration by weight of at least 1%, at least 2%, at least 3%, at least 4%, at least 5%, at least 6%, at least 7%, at least 8%, or at least 9%. In particular embodiments, the food product includes a bone broth protein at a concentration by weight of at least 1%, at least 2%, at least 3%, at least 4%, at least 5%, at least 6%, at least 7%, at least 8%, or at least 9%. In particular embodiments, the food product includes a bone broth protein at a concentration by weight of at least 3.3%, at least 3.4%, at least 3.5%, at least 4%, at least 4.1%, at least 4.2%, at least 4.3%, at least 4.4%, at least 4.5%, at least 4.6%, at least 4.7%, at least 4.8%, at least 4.9%, at least 5.0%, at least 5.1%, at least 5.5%, or at least 6%.

(i-d) Additional Protein Sources. The protein mixture described herein can include alternative or additional proteins. Any protein source known by those skilled in the art can be used within the protein mixture. In particular embodiments, the protein mixture can include casein protein, soy protein, egg protein, pea protein, wheat protein, hemp protein, or rice protein.

Casein is one of the two types of protein found in milk, the other being whey. Casein separates from milk when milk is curdled, a process commonly carried out in the manufacturing of cheese, and is commonly called caseinate, having lost its typical micellar structure. Casein tends to form a gel in the stomach, which slows the digestion. This makes casein an ideal protein source to release protein into the bloodstream over a period of time, e.g., during sleep. Casein also has a high glutamine content, which is a conditionally essential amino acid, necessary for repair of muscle tissue after strenuous exercise and important for gut and immune function. Casein has a relatively low cysteine content which can be compensated by adding other proteins like vegetable proteins. Cysteine is important for the endogenous synthesis of glutathione and therefore plays an important role to protect damage from free radicals. Like many other nutritional compounds, casein is typically bound to a metal ion since the molecule is more stable this way. Specifically, casein is most commonly bound to calcium (Ca2+), sodium (Na+), potassium (K+), or magnesium (Mg2+), since these ions are found naturally in milk and tend to stick to the casein during the extraction process. Nutritionally, these compounds are interchangeable, as all forms of casein are effective protein sources. Micellar casein refers to casein in the form of native micelles. It is a high quality milk protein and naturally occurring in milk in a concentration of 2.6 g/100 ml (Dairy Science and Technology, Walstra et al., CRC Press, 2006). It is concentrated by a process that does not or does not substantially denature the casein proteins and it is marketed as Micellar Casein Isolate (MCI). Fresh skim milk is subjected to a microfiltration process, in much the same process used to concentrate whey protein, to produce a pure, substantially undenaturated milk protein with its native structure. The resulting material contains between 90% and 95% by weight of micellar casein, the rest mainly being whey protein and other non-protein nitrogen and other constituents, such as lactose and inorganic salts such as particular calcium phosphate. Within the context of this disclosure, it is understood that micellar casein may also be provided by other milk protein sources, such as, for instance, sources with essentially preserve the natural 80:20 ratio of casein to whey, such as Milk Protein Concentrate (MPC), Milk Protein Isolate (MPI), and skimmed concentrated milk. MPC has an average protein content of 80 weight % and is a powder product usually prepared by ultrafiltration. MPI has an average protein content of more than 85 weight %. MPI is a powder product usually prepared by precipitation. Within the context of this invention, with the term “casein” both caseinate and micellar casein is indicated.

Soy protein has been used since 1959 as an ingredient for its functional properties in a variety of foods such as salad dressings, soups, vegetarian foods and meat imitations. Its functional properties are emulsification and texturizing. Recently, the popularity of soy protein is increasing, mainly because of its health benefits. For example, health claims have been made for improving heart health (cholesterol reduction), improving bone health (increased bone density), menopausal symptom relief (reduced hot flashes), performance nutrition (faster muscle recovery) and weight management (satisfying hunger). Soy protein is a vegetable protein that contains the essential amino acids in a relatively high proportion for human health. Soy protein is categorized as a high-quality, complete protein although the methionine level is slightly below the WHO 2007 recommendation for methionine content. Soy proteins can be divided into different categories according to their production method. Soy protein isolate (SPI) typically contains 90% protein and is the most refined form of soy protein and is mainly used in meat products to improve texture. Soy protein concentrate (SPC) is basically soybean without the water-soluble carbohydrates and typically contains 70% protein. Textured soy protein (TSP) is made from soy protein concentrate by giving it some texture. TSP typically contains 70% protein and is available as dry flakes or chunks. It will keep its structure when hydrated. Hydrated textured soy protein chunks have a texture similar to ground beef. It can be used as a meat replacement or can be added to meat.

Egg protein is an excellent source of protein and eggs have the highest protein digestibility-corrected amino acid score (PDCAAS), which is used to measure a protein's quality and digestibility. Egg protein is considered a complete protein and is high in leucine, the BCAA that play a large role in muscle health. In particular embodiments, egg protein includes whole egg protein and egg white protein. Egg protein powder can be prepared by dehydrating and pasteurizing the eggs. If the egg protein is egg white protein powder, the egg yolk is removed prior to dehydrating and pasteurizing.

Pea protein is relatively cheap and is added to the mixture to increase the protein content while keeping costs quite low. Pea protein is generally tolerated well by most people, it is lactose-free and is not a common allergen. Pea protein is quite high in cysteine content and can therefore compensate the inadequate amount of cysteine in casein proteins. Furthermore, pea protein is quite high in arginine compared to casein, soy or whey protein which is required for muscle metabolism and which facilitates the intake of body mass while reducing body fat; and it is quite high in lysine, when compared to the vegetable proteins, which is needed to build protein muscle and assist in the maintenance of lean body mass. Pea protein sources can originate from green pea, cowpea, chickpea, and field pea. As a powder, pea protein has a poor taste and it does not mix well, leaving a grainy texture to the protein.

Wheat protein is a plant-based protein. Wheat protein isolate refers to the wheat protein that has been separated from the other flour components, such as starch, fat, fiber, and simple sugars, found in the wheat kernel. The wheat protein can be separated by physical or chemical means. Commercial powdered wheat protein isolates usually have 90 weight % protein.

Hemp protein is a plant-based protein rich in beneficial omega-3 fatty acids and several essential amino acids. It is not, however, considered a complete protein because it has very low levels of the amino acids lysine and leucine.

Rice protein is a protein take from rice. Like other plant-based proteins, it is not a complete protein. Rice protein does include all 9 essential amino acids but it does not reach the adequate amount of lysine to be considered complete. Lysine is essential when it comes to promoting calcium absorption and retention, along with collagen. Rice protein has a distinct flavor; it is relatively sweet compared to other protein powders. In particular embodiments, the rice protein includes a brown rice protein.

(ii) Adaptogens. In particular embodiments, the food product described herein includes adaptogens. Adaptogens are herbs and plants that, when consumed, can help the body adapt to various stresses. Adaptogens also exhibit overlapping pharmacological pleiotropy. While each type of adaptogen has unique properties and benefits, adaptogenic substances generally have the capacity to normalize body functions and strengthen systems compromised by stress. Adaptogens have protective effects on health against a wide variety of environmental assaults, as well as both physical and psychological stressors. Herein, an adaptogen also includes ingredients that do not meet the full requirements to be classified as an adaptogen but are thought of as secondary and supporting adaptogens due to wide-ranging positive effects. In other words, an adaptogen includes any ingredient with adaptogenic effects.

Adaptogens affect the hypothalamic-pituitary-adrenal (HPA axis) in response to stimulation by external stress. Chronic stress exposure, due to varying stressors, often results in HPA axis dysregulation in a wide variety of individuals. HPA axis dysfunction manifests in a variety of ways as symptoms are often non-specific and may include, but are not limited to, weakness, fatigue, malaise, nausea, abdominal pain, poor weight gain, headache, anergia, disturbed sleep, anxiety, immunosuppression, adrenal suppression, hypocortisolism, adrenocorticotropic hormone (ACTH) suppression, and changes in body weight.

Adaptogens are known for their ability to maintain or recover homeostasis and allostasis in the body, as well as promote anabolic recovery. Adaptogens can produce positive responses to stress through regulation of key mediators of the stress response such as, stress-activated c-Jun N-terminal protein kinase (JNK1), β-endorphin, nitric oxide and HPA-axis, including cortisol and glucocorticoid receptors. See, e.g., Panossian A, Wikman G. Pharmaceuticals (Basel). 2010; 3 (1): 188-224. The HPA axis maintains physiological homeostasis and as such regulates many bodily processes thus, adaptogens exhibit polyvalent beneficial effects against chronic inflammation, atherosclerosis, neurodegenerative cognitive impairment, metabolic disorders, cancer, and other aging-related diseases. See, e.g., Panossian, A. (2017). Ann. N.Y. Acad. Sci., 1401:49-64.

Adaptogens exhibit multi-target action and the shared use of several receptors, including corticosteroid, mineralocorticoid, progestin, estrogen, serotonin (5-HT), N-methyl-d-aspartate, and nicotinic acetylcholine, receptor tyrosine kinases, and many G protein-coupled receptors. See, e.g., Panossian, A. (2017). Ann. N.Y. Acad. Sci., 1401:49-64.

In particular embodiments, the food product includes an adaptogen. Examples of adaptogens include mushrooms (e.g., Reishi, Lion's mane, cordyceps, Chaga, Turkey tail, Maitake, Shiitake), holy basil (Tulsi), ashwaganda, Rhodiola Rosea, Turmeric (Curcumin), ginseng, Shcisandra, Astragalus, Goji berry, Moringa, Licorice root, Dangshen, Eleuthero, or Jiaogulan.

In particular embodiments, the adaptogen includes a mushroom blend. In particular embodiments, the mushroom blend includes Reishi, Lion's mane, cordyceps, Chaga, Turkey tail, Maitake, and/or Shiitake. In particular embodiments, the mushroom blend includes Reishi, Lion's mane, cordyceps, Chaga, and/or Turkey tail. Reishi provides calming, stress-relief effects and immune modulation. Lion's Mane supports cognitive health and focus. Cordyceps boost energy and endurance, adding metabolic support. Chaga are rich in antioxidants, support cellular health, and reduce oxidative stress. Turkey Tail contain beta-glucans that strengthen immune function. In particular embodiments, the mushroom blend is in a tomato base or tomato sauce, providing acidity enhancing bioavailability and stability of the mushroom blend. In particular embodiments, the mushroom blend is 0-7 weight % of the composition. In particular embodiments, the mushroom blend is 0, 1, 2, 3, 4, 5, 6, or 7 weight % of the composition. In particular embodiments, the mushroom blend is 0-100 weight % of the adaptogens. In particular embodiments, the mushroom blend is 2-70 weight % of the adaptogens. In particular embodiments, the mushroom blend is 10-30 weight % of the adaptogens. In particular embodiments, the mushroom blend is 0, 2, 4, 6, 8, 10, 12, 14, 16, 18, 20, 22, 24, 25, 27 30, 32, 35, 40, 45, 50, 55, 60, 63, 65, 70, 75, 80, 85, 90 weight % of the adaptogens.

In particular embodiments, the adaptogen includes holy basil (Tulsi). Holy basil is known for its stress-relieving and anti-inflammatory effects, holy basil supports immunity and resilience. In particular embodiments, holy basil is incorporated into the food product using a staged cooking process. In particular embodiments, the staged cooking process preserves the potency of holy basil's heat-sensitive volatile oils. In particular embodiments, holy basil's calming, anti-inflammatory effects balance the cognitive and immune support provided by a mushroom blend. In particular embodiments, the holy basil is 0-7 weight % of the composition. In particular embodiments, the holy basil is 0, 1, 2, 3, 4, 5, 6, or 7 weight % of the composition. In particular embodiments, the holy basil is 0-100 weight % of the adaptogens. In particular embodiments, the holy basil is 2-70 weight % of the adaptogens. In particular embodiments, the holy basil is 10-30 weight % of the adaptogens. In particular embodiments, the holy basil is 0, 2, 4, 6, 8, 10, 12, 14, 16, 18, 20, 22, 24, 25, 27 30, 32, 35, 40, 45, 50, 55, 60, 63, 65, 70, 75, 80, 85, 90 weight % of the adaptogens.

In particular embodiments, the adaptogen includes ashwaganda. Ashwagandha reduces cortisol levels, enhances resilience to stress, and supports energy and endurance. In particular embodiments, ashwagandha is added later in the cooking process to protect its active compounds from heat degradation, allowing for maximum adaptogenic potency. In particular embodiments, ashwagandha's adaptogenic properties harmonize with reishi and holy basil, while the acidic tomato environment aids in absorbing its active withanolides. In particular embodiments, the ashwaganda is 0-7 weight % of the composition. In particular embodiments, the ashwaganda is 0, 1, 2, 3, 4, 5, 6, or 7 weight % of the composition. In particular embodiments, the ashwaganda is 0-100 weight % of the adaptogens. In particular embodiments, the ashwaganda is 2-70 weight % of the adaptogens. In particular embodiments, the ashwaganda is 10-30 weight % of the adaptogens. In particular embodiments, the ashwaganda is 0, 2, 4, 6, 8, 10, 12, 14, 16, 18, 20, 22, 24, 25, 27 30, 32, 35, 40, 45, 50, 55, 60, 63, 65, 70, 75, 80, 85, 90 weight % of the adaptogens.

In particular embodiments, the adaptogen includes Rhodiola Rosea. Rhodiola Rosea (i.e., Rhodiola) improves endurance, mental clarity, and reduces fatigue, supporting cognitive and physical performance. In particular embodiments, Rhodiola's sensitive compounds, such as rosavins, benefit from a later-stage addition, ensuring they remain effective. In particular embodiments, Rhodiola's energy-boosting and cognitive-support effects pair well with cordyceps and lion's mane. In particular embodiments, the Rhodiola Rosea is 0-7 weight % of the composition. In particular embodiments, the Rhodiola Rosea is 0, 1, 2, 3, 4, 5, 6, or 7 weight % of the composition. In particular embodiments, the Rhodiola Rosea is 0-100 weight % of the adaptogens. In particular embodiments, the Rhodiola Rosea is 2-70 weight % of the adaptogens. In particular embodiments, the Rhodiola Rosea is 10-30 weight % of the adaptogens. In particular embodiments, the Rhodiola Rosea is 0, 2, 4, 6, 8, 10, 12, 14, 16, 18, 20, 22, 24, 25, 27 30, 32, 35, 40, 45, 50, 55, 60, 63, 65, 70, 75, 80, 85, 90 weight % of the adaptogens.

In particular embodiments, the adaptogen includes Turmeric (Curcumin). Turmeric is a powerful anti-inflammatory and antioxidant. Turmeric supports immune health, cellular protection, and resilience. Curcumin is more heat-stable and can be added earlier in the cooking process to allow it to integrate fully, creating a balanced and shelf-stable anti-inflammatory foundation. In particular embodiments, turmeric is added early in the cooking process. In particular embodiments, curcumin's anti-inflammatory effects work well with lycopene in tomatoes, providing a dual anti-inflammatory support beneficial for managing stress-related inflammation or physical recovery. In particular embodiments, the curcumin is 0-7 weight % of the composition. In particular embodiments, the curcumin is 0, 1, 2, 3, 4, 5, 6, or 7 weight % of the composition. In particular embodiments, the curcumin is 0-100 weight % of the adaptogens. In particular embodiments, the curcumin is 2-70 weight % of the adaptogens. In particular embodiments, the curcumin is 10-30 weight % of the adaptogens. In particular embodiments, the curcumin is 0, 2, 4, 6, 8, 10, 12, 14, 16, 18, 20, 22, 24, 25, 27 30, 32, 35, 40, 45, 50, 55, 60, 63, 65, 70, 75, 80, 85, 90 weight % of the adaptogens.

In particular embodiments, the adaptogen includes a mushroom blend, holy basil, ashwaganda, Rhodiola Rosea, and/or curcumin. In particular embodiments, adaptogens include phenolic compounds or tetracyclic triterpenoids. In particular embodiments, the mushroom blend includes Reishi, Lion's mane, cordyceps, Chaga, and/or Turkey tail.

Phenolic compounds include, but are not limited to, phenylpropanoids and phenylethane derivatives, such as salidroside (rhodioloside), rosavin, syringin, triandrin, tyrosol, and lignans, such as eleutherosid E and schisandrin B. These compounds are structurally similar to the catecholamines, which mediate the sympathoadrenal system involved in activation of the stress system in the early stages of a stress response.

Tetracyclic triterpenoids include, but are not limited to, cucurbitacin R diglucoside, ginsenosides, and phytosterol-glycosides (e.g., eleutheroside A, sitoindosides, and daucosterol). These compounds structurally resemble the corticosteroids that act as stress hormones involved in the protective inactivation of the stress system.

In particular embodiments, the food product includes an adaptogen at a concentration by weight of 0.01-7%. In particular embodiments, the food product includes an adaptogen at a concentration by weight of 0.03-6%. In particular embodiments, the food product includes an adaptogen at a concentration by weight of 0.05-5%. In particular embodiments, the food product includes an adaptogen at a concentration by weight of at least 0.1%, at least 1%, at least 2%, at least 3%, at least 4%, at least 5%, at least 6%, at least 7%, at least 8%, or at least 9%.

In particular embodiments, the food product includes one or more adaptogens. In particular embodiments, a ratio of a first adaptogen to a second adaptogen includes 1:0, 1:1, 2:1, 3:1, 4:1, 5:1, 6:1, 7:1, 8:1, 9:1, or 10:1.

(iii) Probiotics. In particular embodiments, the food product described herein can include probiotics. Probiotics are live microorganisms that are intended to have health benefits when consumed or applied to the body. Probiotics are commonly found in yogurt and other fermented foods, dietary supplements, and beauty products. In particular embodiments, the food product includes a probiotic including at least one non-pathogenic microorganism. Example microorganisms include Lactobacilli, Bifidobacteria, Streptococci, Pediococci, Leuconostoc, propionic and acetic bacteria. Among the Lactobacilli include but are not limited to Lactobacillus acidophilus, Lactobacillus bifidus, Lactobacillus brevis, Lactobacillus bulgaricus, Lactobacillus delbrucki, Lactobacillus casei, Lactobacillus cellobiosus, Lactobacillus fermentum, Lactobacillus gasseri, Lactobacillus germentum, Lactobacillus helveticus, Lactobacillus johnsonhii, Lactobacillus lactis, Lactobacillus leichimanii, Lactobacillus plantarum, Lactobacillus reuteri, Lactobacillus rhamnosus, Lactobacillus sake, Lactobacillus salivaroes, Lactobacillus thermophilus and Lactobacillus xylosus. Among the Bifidobacteria are those including but not limited to Bifidobacterium adolescentis, Bifidobacterium bifidum, Bifidobacterium breve, Bifidobacterium cereus, Bifidobacterium infantis, Bifidobacterium lactis, Bifidobacterium longum, and Bifidobacterium thermophilus. Among the Streptococci bacteria are those including but not limited to Streptococcus lactis, Streptococcus cremoris, Streptococcus diacetylactis, Streptococcus thermophilus, and Streptococcus faecium. In particular embodiments, one or more cultures of any of these strains of microorganism or another probiotic, non-pathogenic microorganism may be employed. In one embodiment, the microorganisms used include the Lactobacillus acidophilus strain and/or the Lactobacillus heveticus strain. In particular embodiments, the probiotic includes strains of Lacobacillus and/or Bifidobacterium. In particular embodiments, the probiotic is in a dry, viable form.

Storage temperatures for the probiotic cultures include 39° F. to 46° F., but can vary with the culture selected. The cultures can be prepared by freeze drying and can be effectively stored as recommended by the manufacturer. Among the commercial sources are DSM®, Dansico®, Institut Rossell-Lallemand, and others.

In particular embodiments, the food product includes at least 10⁴ colony forming units (CFU) per serving, at the end of shelf life. Colony-forming unit (CFU) is a measure of viable bacterial numbers. Unlike in direct microscopic counts where all cells, dead and living, are counted, CFU measures viable cells. In one format, a sample is spread or poured on a surface of an agar plate, left to incubate and the number of colonies formed are counted. CFU is not an exact measure of numbers of viable cells, as a colony-forming unit may contain any number of cells. A “serving,” as used herein, refers to a single portion of food or a helping as customarily taken at a particular time. Considerations for CFU include choosing a suitable dosage form and its effect on flavor.

In particular embodiments, the probiotic can also include contain quantities of prebiotics. Prebiotics are substances, typically not digested in the stomach of the host, which promote the growth of certain intestinal bacteria. Prebiotics which may be included in the food products described include sugars, such as lactose or tagatose, and dietary fiber. Other prebiotics known in the art may also be included.

In particular embodiments, the food product of the present disclosure can be formulated with dietary fiber as a prebiotic and can accomplish this without significant decrease in stability. The fiber can contribute to the healthfulness of the food product without diminishing the stability of the probiotics. The result is a food product that helps to promote a healthy intestinal bacterial flora. The fiber can be provided from any suitable source, including that derived from various vegetable and fruit sources, including purified vegetable fiber, such as insulin extracted from the roots of the chicory plant; various fruit pulps and vegetable pulps available largely as byproducts of juicing or other food preparation operations, such as apple pulp, citrus pulp, mango pulp, grape pulp, cranberry pulp and beet pulp; and fruit and vegetable purees, such as those prepared from fresh and/or frozen fruits and vegetables. Other sources and considerations regarding fiber mixtures are described elsewhere herein.

In particular embodiments, the probiotics are included in the food product at 10⁴ to 10¹² colony forming units (CFU), at 10⁶ to 10¹¹ CFU, or at 10⁷ to 10⁹ CFU per serving. In particular embodiments, the probiotics are included in the food product at 10⁴, 10⁵, 10⁶, 10⁷, 10⁸, 10⁹, 10¹⁰, 10¹¹, or 10¹² CFU per serving. In particular embodiments, a serving is 50-500 g, 75-250 g, or 100-150 g. In particular embodiments, a serving is 100-150 g. Different probiotic strains may require varying CFU levels for efficacy. Storage and degradation are also considered in determining a starting CFU.

In particular embodiments, the food product includes probiotics at a concentration by weight of 0-10%. In particular embodiments, the food product includes probiotics at a concentration by weight of 0.01-5%. In particular embodiments, the food product includes probiotics at a concentration by weight of 0.05-3%. In particular embodiments, the food product includes probiotics at a concentration by weight of 0.1-1%. In particular embodiments, the food product includes probiotics at a concentration by weight of at least 0.01%, at least 0.05%, at least 0.1%, at least 0.3%, at least 0.5%, at least 1%, or at least 1.2%. For some strains, a carrier (e.g., maltodextrin) is required for efficacy and the amount of probiotics may be increased.

(iv) Fiber Mixture. In particular embodiments, the food product described herein can include a fiber mixture. Diets that are high in fiber are considered to promote improved heart health. Food sources that are high in fiber include split peas, lentils, black beans, artichokes and raspberries. Dietary fiber can be divided into two types: soluble fiber (which dissolves in water) and insoluble fiber (which does not dissolve in water). In particular embodiments, the food product includes a soluble fiber and/or an insoluble fiber.

Soluble fiber tends to form a gel during digestion and will actually slow the digestive process. Slowing digestion can help regulate blood sugar. Soluble fiber can also help reduce LDL (bad) cholesterol. Soluble fibers can be found in chicory root, tapioca, corn oats, peas, beans, apples, bananas, avocados, citrus fruits, carrots, barley, and psyllium. In particular embodiments, the soluble fiber has a sweet flavor. In particular embodiments, the soluble fiber includes chicory root. In particular embodiments, the soluble fiber includes tapioca fiber.

Insoluble fiber does not dissolve in water and is not digestible by humans. Insoluble fiber adds bulk to the stool causing it to move through the digestive system quickly, which relieves constipation. Insoluble fiber helps to balance the pH in the intestines and helps prevent colon cancer. Natural sources of insoluble fiber are seeds, nuts, dark green leafy vegetables, and wheat bran. In particular embodiments, the insoluble fiber is derived from vegetables and/or grains (e.g., whole grains, wheat).

The fiber mixture can play an important role in the food product by functioning as a sweetener, thereby lowering the need for added sugars or sweeteners. The fiber mixture also provides thickness to the food product which can be balanced to provide a desirable texture and consistency. Through these features, the fiber mixture provides the benefits of high fiber and low net carbohydrates resulting in benefits for digestive health, balanced blood sugar, and satiety along with balanced flavor and desirable texture and consistency.

The terms “added sugars or sweeteners” refers to the saccharides or other caloric sweeteners that are intentionally introduced into a composition during its formulation. This excludes sugars that are inherently present in natural ingredients such as vegetables, fruits, or other ingredients.

In particular embodiments, the food product includes a fiber mixture at a concentration by weight of 0-10%. In particular embodiments, the food product includes a fiber mixture at a concentration by weight of 0-8%. In particular embodiments, the food product includes a fiber mixture at a concentration by weight of 0-6%. In particular embodiments, the food product includes a fiber mixture at a concentration by weight of at least 0.1%, at least 1%, at least 2%, at least 3%, at least 4%, at least 5%, at least 6%, at least 7%, at least 8%, or at least 9%.

In particular embodiments, the fiber mixture is 100% soluble fiber. In particular embodiments, the ratio of soluble fiber to insoluble fiber includes 1:0, 1:1, 2:1, 3:1, 4:1, 5:1, 6:1, 7:1, 8:1, 9:1, or 10:1. In particular embodiments, the insoluble fiber includes the fiber already within the food base. In particular embodiments, the fiber mixture includes added fiber (e.g., soluble fiber).

(v) Food Base. The food base refers to a comestible ingredient of which, the protein mixture is added to in order to elevate the protein content. In particular embodiments, collagen peptide, whey protein, bone broth protein, and adaptogens are added to a food base to provide a food product, wherein the food product has a high protein content. A food base can be any comestible component including sauces, candy, food bars, pastries, cheese, rice cakes, granola, cereal, meat, dressings, soups, potatoes, etc. In particular embodiments, the food base is a sauce. The term “sauce” as used herein describes a thick liquid served with food or in food to add moisture and flavor. A sauce includes dressing, condiments, dips, jus, relish, and salsa. In particular embodiments, the sauce is a tomato-based sauce such as pasta sauce, taco sauce, barbecue sauce, soup base, chili sauce, hot sauce, Indian-style sauce, or salsa. An Indian-style sauce can include any sauce typically found in Indian cuisine such as curry, masala, or chutney. In particular embodiments, the food base includes a pasta sauce.

As will be understood by one of skill in the art, the food base can contain a number of ingredients derived from animals or plants. For example, the food base may include a plant material (e.g., tomato) as well as seasoning and spices. The term “tomato” as used herein includes the fruit in several forms, such as raw tomatoes, crushed tomatoes, processed tomatoes, tomato paste, tomato puree, and the like.

The term “seasonings” includes condiments for enhancing the flavor of food products, primarily chopped vegetables such as onion, garlic, bell pepper, carrots, celery, and the like, as well as conventional seasonings such as salt, pepper, sugar, and others. These may be in dehydrated, powdered, oleoresin, or other conventional forms well known in the art.

The term “spices” includes various aromatic plants and herbs used to enhance the flavor of foods, such as oregano, basil, bay leaf, parsley, and others well known in the art. These may be in flaked, dehydrated, powdered, oleoresin, or other conventional known forms.

In addition to the animal or plant material, seasonings, and spices, the food base can include common raw materials and additives. Examples of the common raw materials and additives include the above-described aromatic materials, common salts, sugars (sugar, fructose, glucose, starch syrup, isomerized glucose syrup, etc.), umami seasonings (protein hydrolysate, yeast extract, etc.), amino acid-based seasonings (glycine, alanine, sodium glutamate, etc.), nucleic acid-based seasonings (sodium inosinate, sodium guanylate, etc.), thickening agents (gums such as xanthan gum, locust gum or carrageenan, starches such as wheat starch or corn starch, etc.), acidulants (citric acid, lactic acid, malic acid, tartaric acid, etc.), antioxidants (tocopherol, cysteine hydrochloride, etc.), taste improvers (for example, a bitter taste suppressor, etc.), pH adjusters, ethanol, edible fat and oil, and coloring agents. The additive amount of such a raw material or additives may be adjusted, as appropriate, depending on the desired quality.

In particular embodiments, the food base includes 0 g-15 g of protein per gram of food base. In particular embodiments, the food base includes at least 0 g, at least 1 g, at least 2 g, at least 3 g, at least 4 g, at least 5 g, at least 6 g, at least 7 g, at least 8 g, at least 9 g, at least 10 g, at least 11 g, at least 12 g, at least 13 g, or at least 14 g of protein per gram of food base. In particular embodiments, the food base includes at least 0.010 g, at least 0.011 g, at least 0.012 g, at least 0.013 g, at least 0.014 g, at least 0.015 g, at least 0.016 g, at least 0.017 g, at least 0.018 g, at least 0.019 g, or at least 0.020 g of protein per gram of food base. In particular embodiments, the food base includes 0.016 g of protein per gram of food base. In particular embodiments, the food base includes 0% fat-25% fat. In particular embodiments, the food base includes at least 0% fat, at least 1% fat, at least 2% fat, at least 3% fat, at least 4% fat, at least 5% fat, at least 6% fat, at least 7% fat, at least 8% fat, at least 9% fat, at least 10% fat, at least 15% fat, or at least 20% fat. In particular embodiments, the food base includes 2.8% fat. In particular embodiments, the food base is a pasta sauce.

In particular embodiments, the food product includes a food base at a concentration by weight of 50-99%. In particular embodiments, the food product includes a food base at a concentration by weight of 70-90%. In particular embodiments, the food product includes a food base at a concentration by weight of 75-85%. In particular embodiments, the food product includes a food base at a concentration by weight of at least 50%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, or at least 94%.

(vi) Edible Acid. One method of preparing the composition such that it is shelf-stable is to adjust the pH of the food product to be acidic. In particular embodiments, the pH is adjusted by the addition of edible acids. Furthermore, edible acids used in food products disclosed herein can serve any one or more of several functions, including for example, acting as a mild preservative by providing microbiological stability, providing antioxidant activity, lending tartness to the taste, enhancing palatability, and modifying sweetness.

An edible acid refers to a substance having a pH less than 7 that is suitable to be eaten by humans. Edible acids include citric acid, vinegar, malic acid, tartaric acid, acetic acid, benzoic acid, butyric acid, lactic acid, fumaric acid, succinic acid, ascorbic acid, adipic acid, phosphoric acid, and mixtures thereof. In particular embodiments, a level of inorganic acids can also be used to lower the pH including phosphoric acid, carbonic acid, sodium hydrogen phosphate, and/or anhydric chloric acid. The type, amount, and combination of acid added to reach the desired pH can have an effect on the product such as changing the flavor of the product. For example, acidification with citric acid provides more sourness and sharpness than acidification with hydrochloric acid at a given pH. Acetic acid can add a vinegary flavor to foods.

As used herein, “taste” refers to the overall perception of a balance of sweet-tart-salty quality of the comestible, including a combination of sweetness-tartness-saltiness flavor perception, temporal effects of the flavor perception (e.g., bitterness and metallic), residual perception (aftertaste), and tactile perception (e.g., body and thickness).

Determination of the specific pH and acid level of any particular product can readily be done by standard analytical techniques. Alternatively, the acid level can be calculated in advance by adding together the acid contents of each of the ingredients of the food product, along with any acids added as part of the formulation. Acid can be added as needed or excess acid can be neutralized as required to obtain the desired pH. In particular embodiments, the acid can be added in dry or liquid form.

In particular embodiments, the pH of either the food base, the adaptogens, or protein mixture can vary from batch to batch, thus the amount of edible acid needed to reach the desired PH level may vary.

In particular embodiments, the food product (e.g., sauce) includes an edible acid at a concentration by weight of 0-2%. In particular embodiments, the food product (e.g., sauce) includes an edible acid at a concentration by weight of 0.1-1.5%. In particular embodiments, the food product includes an edible acid at a concentration by weight of at least 0.1%, at least 0.2%, at least 0.3%, at least 0.4%, at least 0.5%, at least 0.6%, at least 0.7%, at least 0.8%, at least 0.9%, at least 1.0%, at least 1.1%, at least 1.2%, at least 1.3%, at least 1.4%, at least 1.5%, at least 1.6%, at least 1.7%, at least 1.8%, or at least 1.9%. In particular embodiments, the food product includes an edible acid at a concentration by weight of at least 0.50%, at least 0.51%, at least 0.52%, at least 0.53%, at least 0.54%, at least 0.55%, at least 0.56%, at least 0.57%, at least 0.58%, at least 0.59%, at least 0.8%, at least 0.99%, at least 1.1%, or at least 1.2%. In particular embodiments, the food product includes an edible acid at a concentration by weight of 1.2%.

The use of edible acids to balance the alkaline nature of, for example, hydrolyzed proteins provides a safe, acidic environment such that the food product is shelf-stable without sacrificing taste. Balancing proteins' higher pH with edible acids provides both flavor retention and microbial safety, essential for high-protein food products.

(vii) Composition of Food Product. The components described herein are combined to prepare an adaptogenic, high-protein composition (i.e., food product), such as an adaptogenic, high-protein sauce. In particular embodiments, the composition includes any combination of proteins to create a protein mixture. In particular embodiments, the composition includes a collagen peptide, a whey protein, a bone broth protein, adaptogens, and a food base. In particular embodiments, the composition further includes an edible acid.

In particular embodiments, the composition includes a protein mixture at a concentration by weight of 1-40%, adaptogens at a concentration by weight of 0-10%, and a food base at a concentration by weight of 25-99%, wherein the selected amounts do not exceed 100%. In particular embodiments, the composition includes a protein mixture at a concentration by weight of 1-40%, adaptogens at a concentration by weight of 0-10%, and a food base at a concentration by weight of 25-99%, and an edible acid at a concentration by weight of 0-5%, wherein the selected amounts do not exceed 100%.

In particular embodiments, the composition includes a protein mixture at a concentration by weight of 1-25%, adaptogens at a concentration by weight of 0.01-7%, and a food base at a concentration by weight of 50-99%, wherein the selected amounts do not exceed 100%. In particular embodiments, the composition includes a protein mixture at a concentration by weight of 1-25%, adaptogens at a concentration by weight of 0.01-7%, and a food base at a concentration by weight of 50-99%, and an edible acid at a concentration by weight of 0.1-2%, wherein the selected amounts do not exceed 100%. In particular embodiments, the protein mixture includes any combination of proteins.

In particular embodiments, the composition includes a protein mixture at a concentration by weight of 5-20%, adaptogens at a concentration by weight of 0.03-6%, and a food base at a concentration by weight of 70-90%, wherein the selected amounts do not exceed 100%. In particular embodiments, the composition includes a protein mixture at a concentration by weight of 5-20%, adaptogens at a concentration by weight of 0.03-6%, and a food base at a concentration by weight of 70-90%, and an edible acid at a concentration by weight of 0.3-1.5%, wherein the selected amounts do not exceed 100%.

In particular embodiments, the composition includes a protein mixture at a concentration by weight of 10-18%, adaptogens at a concentration by weight of 0.05-5%, and a food base at a concentration by weight of 75-85%, wherein the selected amounts do not exceed 100%. In particular embodiments, the composition includes a protein mixture at a concentration by weight of 10-18%, adaptogens at a concentration by weight of 0.05-5%, and a food base at a concentration by weight of 75-85%, and an edible acid at a concentration by weight of 1.2%, wherein the selected amounts do not exceed 100%.

In particular embodiments, the protein mixture includes a collagen peptide, a whey protein, and a bone broth protein.

In particular embodiments, the composition includes a collagen peptide at a concentration by weight of 0-25%, a whey protein at a concentration by weight of 0-25%, a bone broth protein at a concentration by weight of 0-25%, adaptogens at a concentration by weight of 0.01-7%, and a food base at a concentration by weight of 70-90%, wherein the selected amounts do not exceed 100%. In particular embodiments, the composition includes a collagen peptide at a concentration by weight of 0-25%, a whey protein at a concentration by weight of 0-25%, a bone broth protein at a concentration by weight of 0-25%, adaptogens at a concentration by weight of 0.01-7%, and a food base at a concentration by weight of 70-90%, and an edible acid at a concentration by weight of 0.3-1.5%, wherein the selected amounts do not exceed 100%.

In particular embodiments, the composition comprises a mushroom blend at a concentration by weight of 0-7%, holy basil at a concentration by weight of 0-7%, ashwaganda at a concentration by weight of 0-7%, Rhodiola Rosea at a concentration by weight of 0-7%, and/or curcumin at a concentration by weight of 0-7%, wherein the selected amounts do not exceed 7%. In particular embodiments, the composition comprises a mushroom blend at a concentration by weight of 0-5%, holy basil at a concentration by weight of 0-5%, ashwaganda at a concentration by weight of 0-5%, Rhodiola Rosea at a concentration by weight of 0-5%, and curcumin at a concentration by weight of 0-5%, wherein the selected amounts do not exceed 7%.

In particular embodiments, the composition comprises Reishi at a concentration by weight of 0-7%, Lion's mane at a concentration by weight of 0-7%, cordyceps at a concentration by weight of 0-7%, Chaga at a concentration by weight of 0-7%, and/or Turkey tail at a concentration by weight of 0-7%, wherein the selected amounts do not exceed 7%. In particular embodiments, the composition comprises Reishi at a concentration by weight of 0-5%, Lion's mane at a concentration by weight of 0-5%, cordyceps at a concentration by weight of 0-5%, Chaga at a concentration by weight of 0-5%, and/or Turkey tail at a concentration by weight of 0-5%, wherein the selected amounts do not exceed 7%.

In particular embodiments, the composition includes additional additives at a concentration by weight of less than 20%, less than 5%, less than 1%, less than 0.1%, less than 0.01%, or less than 0.001%. In particular embodiments, the additional additives includes a taste-masking agent. In particular embodiments, the taste-masking agent includes a bitter blocker.

In particular embodiments, the ratio of protein mixture to food base includes 14:76, 7:40, 17:76, 17:80, 15:125, 16:125, 17:125, 18:125, 19:125, 20:125, 21:125, 22:125, 23:125, 24:125, 25:125, 26:125, 27:125, 28:125, 29:125, 30:125, 31:125, 32:125, 33:125, 34:125, or 35:125.

In particular embodiments, the ratio of protein mixture to food base to edible acid includes 14:76:1, 7:40:1, 17:76:1, 17:80:1, 14:76:1.2, 24:127:2, 24:123:2, 115:127:3, 115:125:5, 115:123:6, 115:625:4, 24:130:1, 22:121:1, 20:120:1, 21:126:1, 112:126:4, 115:125:3, 114:122:6, or 115:127:5.

In particular embodiments, the ratio of collagen peptide to whey protein to bone broth protein includes 17:1:9, 17:1:8, 17:1:7, 17:1:6, 17:1:5, 16:1:9, 16:1:8, 16:1:7, 16:1:6, 16:1:5, 15:1:9, 15:1:8, 15:1:7, 15:1:6, 15:1:5, 14:1:9, 14:1:8, 14:1:7, 14:1:6, 14:1:5, 13:1:9, 13:1:8, 13:1:7, 13:1:6, 13:1:5, 75:2:35, 75:3:35, 75:4:35, or 75:5:35.

In particular embodiments, the ratio of collagen peptide to whey protein to bone broth protein to food base includes 17:1:9:130, 17:1:8:129, 17:1:7:128, 17:1:6:127, 17:1:5:126, 16:1:9:125, 16:1:8:124, 16:1:7:123, 16:1:6:122, 16:1:5:121, 15:1:9:120, 15:1:8:127, 15:1:7:125, 15:1:6:123, 15:1:5:125, 14:1:9:130, 14:1:8:127, 14:1:7:121, 14:1:6:125, 14:1:5:120, 13:1:9:124, 13:1:8:127, 13:1:7:126, 13:1:6:125, 13:1:5:140, 75:2:35:126, 75:3:35:125, 75:4:35:122, or 75:5:35:127.

In particular embodiments, the ratio of collagen peptide to whey protein to bone broth protein to food base to edible acid includes 17:1:6:127:2, 16:1:7:123:2, 75:5:35:120:4, 75:5:35:127:3, 75:5:35:125:5, 75:5:35:123:6, 75:5:35:625:4, 14:1:9:130:1, 14:1:7:121:1, 14:1:5:120:1, 13:1:7:126:1, 75:2:35:126:4, 75:3:35:125:3, 75:4:35:122:6, or 75:5:35:127:5.

In particular embodiments, the ratio of fiber mixture to protein mixture includes 1:1, 1:2, 1:3, 1:4, 1:5, 1:6, 1:7, 1:8, 1:9, or 1:10. In particular embodiments, the ratio of fiber mixture to protein mixture includes 1:3.

It should be understood that compositions of food products in accordance with this disclosure may have any of numerous different specific formulations or constitutions. The formulation of the composition in accordance with this disclosure can vary to a certain extent, depending upon such factors as the product's intended market segment, desired nutritional characteristics, flavor profile, storage, and the like. For example, it will generally be an option to add further ingredients to the formulation of a particular embodiment. In addition to the components (or ingredients) described herein, additional additives can be incorporated into the composition. For example, vitamins, minerals, flavoring agents, sweeteners, taste-masking agents (e.g., bitter blockers), colorants, and/or food-grade preservatives.

Example vitamins include Vitamin A, vitamin B1, vitamin B2, vitamin B3, vitamin B12, vitamin C, vitamin E, vitamin D, niacin (vitamin PP), biotin (vitamin H), menadione (vitamin K), folic acid, and pyridoxine (B6). Minerals include iron, zinc, calcium, phosphorus, potassium, magnesium, and fluoride. Amino acids include lysine, histidine, isoleucine, leucine, methionine, phenylalanine, threonine, tryptophan, and valine. Exemplary nutritional additives can be found in Roberts et al., Nutraceuticals: The Complete Encyclopedia of Supplements, Herbs, Vitamins, and Healing Foods (American Nutraceutical Association, 2001); in Physicians' Desk Reference for Nutritional Supplements, 1^{st} Ed. (2001); and in The Physicians' Desk Reference for Herbal Medicines, 1^{st} Ed. (2001), which are incorporated by reference.

Example flavoring agents include lemon juice concentrate, tangerine flavor, tangerine concentrate, and/or apple juice concentrate.

Example sweeteners include maltose, sucrose, glucose, fructose, invert sugars and mixtures thereof. These sugars can be incorporated into the aqueous dispersion in solid or liquid form. Fructose can be obtained or provided as liquid fructose, high fructose corn syrup (HFCS), dry fructose or fructose syrup. High fructose corn syrup is commercially available as HFCS-42, HFCS-55 and HFCS-90, which include 42%, 55% and 90%, respectively, by weight of the sugar solids therein of fructose. In particular embodiments, sweeteners can be provided to some extent by other components of the aqueous dispersion, such as by fruit juice, flavorants, and so forth. Sweeteners can be employed in the process of the present disclosure in amounts ranging from 0.01% to 20%, from 6% to 14%, sugar solids by weight of the aqueous dispersion.

Example taste-masking agents include sodium bicarbonate, ion-exchange resins, cyclodextrin inclusion compounds, adsorbates, and the like. A taste-masking agent can include a bitter blocker. In particular embodiments, a bitter blocker includes sodium acetate, sodium gluoconate, or adenosine 5′monophosphate. In particular embodiments, a bitter blocker includes a composition of ingredients. In particular embodiments, a bitter blocker includes a lactose-based ingredient, a spice, and/or a cucumber extract. In particular embodiments, a bitter blocker includes at least two lactose-based ingredients, a spice, and/or a cucumber extract. In particular embodiments, a lactose-based ingredient includes milk, yogurt, cream, condensed milk, cheese (e.g., ricotta cheese), milk powder, whey, condensed milk, or sour cream. In particular embodiments, a spice includes clove, cinnamon, basil, thyme, rosemary, cardamom, cumin, bay leaf, fenugreek, paprika, garlic, ginger, nutmeg, parsley, dill, black pepper, white pepper, or cayenne pepper. In particular embodiments, the food product includes a taste-masking agent. In particular embodiments, the food product includes a bitter blocker.

Colorants are additives such as dye, pigments, or substances the impart color to what they are added to. Food-grade colorants include carotenoids, chlorophyll, anthocyanin, turmeric, paprika, carmine, elderberry juice, lycopene, caramel coloring, and annatto.

Food-grade preservatives include antioxidant preservatives and antimicrobial preservatives. Antioxidant preservatives include ascorbyl palmitate, ascorbic acid, butylated hydroxyanisole (BHA) butylated hydroxytoluene (BHT), citric acid, sulfites, tertiary butylhydroquinone (TBHQ), tocopherols, and salts and mixtures thereof. Antimicrobial preservatives include acetic acid, benzoic acid, potassium sorbate, natamycin, nisin, nitrates, nitrites, propionic acid, sorbic acid, sulfites, sodium hexametaphosphate, sulfur dioxide, and salts and mixtures thereof.

In particular embodiments, the food product includes all natural ingredients. According to the Federal Register, the word “natural” means that nothing artificial or synthetic (including colors regardless of source) is included in, or has been added to, the product that would not normally be expected to be there (56 FR 60421 at 60466).

(viii) Preparation of the Composition. In particular embodiments, a method for producing the composition described herein includes: admixing a protein mixture, adaptogens, and a food base thereby preparing the composition. In particular embodiments, a method for producing the composition described herein includes: admixing a collagen peptide, a whey protein, a bone broth protein, adaptogens, and a food base thereby preparing the composition. In particular embodiments, the method further includes admixing an edible acid to the composition. In particular embodiments, each component (e.g., collagen peptide, whey protein, bone broth protein, adaptogens, food base, etc.) can be admixed simultaneously or sequentially in any order.

In particular embodiments, a method for producing the composition described herein includes: adding components (e.g., adaptogens and/or protein mixture) to a food base; hydrating the components within the food base; and mixing the components and food base.

In particular embodiments, a method for producing the composition described herein includes: hydrating powder components within water; and mixing the hydrated powder components into the composition. Alternatively, a method for producing the composition described herein includes: adding powder components to the composition; hydrating the powder components within the composition; and mixing the food composition.

In particular embodiments, a method for producing the composition described herein includes: mixing the protein mixture, adaptogens, food base, and edible acid to create a food product mixture; heating the food product mixture; and packaging the heated food product mixture.

In particular embodiments, a method for producing the composition described herein includes: mixing the protein mixture, adaptogens, food base, edible acid, and additional additives to create a food product mixture; heating the food product mixture; and packaging the heated food product mixture.

In particular embodiments, the powder components include collagen peptide, whey protein, and/or bone broth protein. In particular embodiments, the powder components include adaptogens. In particular embodiments, the powder components are allowed to hydrate in the food base for at least 1 min, at least 2 min, at least 3 min, at least 5 min, at least 10 min, at least 15 min, at least 20 min, at least 25 min, at least 30 min, at least 1 hour, or at least 2 hours. In particular embodiments, the food base includes water.

In particular embodiments, a method for producing the composition described herein includes cooking ingredients (e.g., adaptogens, protein mixture, food base). In particular embodiments, a method for producing the composition described herein includes cooking adaptogens, protein mixture, and/or food base.

In particular embodiments, to maximize the stability and effectiveness of each adaptogen, the food product utilizes a staged cooking process where ingredients (e.g., adaptogens, protein mixture, etc.) are added at different stages based on their heat sensitivity. This method ensures that each adaptogen retains its beneficial properties without compromising the flavor or integrity of the food product. In particular embodiments, a staged cooking process includes early-stage cooking, mid-stage cooking, and late-stage cooking. In particular embodiments, the staged cooking process includes from start to finish: early-stage cooking, then mid-stage cooking, and then late-stage cooking. In particular embodiments, early-stage cooking includes heat stable ingredients (i.e., early-stage ingredients). In particular embodiments, heat stable ingredients, include turmeric, chaga, and reishi. This allows them to integrate fully into a food base, maximizing their absorption and balancing their flavor. In particular embodiments, mid-stage cooking includes moderately heat-sensitive ingredients (i.e., mid-stage ingredients). In particular embodiments, moderately heat-sensitive ingredients include cordyceps and ashwagandha. These ingredients benefit from mild heat to release their active compounds, but prolonged exposure can degrade their potency. Adding these adaptogens at the mid-stage allows for adequate extraction of their active components without risking thermal degradation. In particular embodiments, late-stage cooking includes sensitive ingredients (e.g., heat sensitive ingredients or late-stage ingredients). In particular embodiments, sensitive ingredients include delicate, volatile compounds such as holy basil, Rhodiola Rosea, Lion's mane, and Turkey tail. This ensures that their active compounds are preserved, maximizing adaptogenic effects.

In particular embodiments, the preparation of the composition includes 1) formulation and ingredient selection, 2) food base preparation, 3) addition of adaptogens to the composition, and 4) packaging. In particular embodiments, the preparation includes cooking ingredients while adding ingredients. In particular embodiments, the addition of adaptogens to the composition includes temperature-controlled adaptogen addition. In particular embodiments, the packaging includes cold-fill packaging and/or airtight sealing. In particular embodiments, the preparation of the composition further includes encapsulation of sensitive ingredients before addition to the composition. In particular embodiments, the preparation of the composition further includes quality control and stability testing.

The food base preparation includes cooking the food base (and in some cases, other ingredients such as the protein mixture, adaptogens, edible acids, etc.). In particular embodiments, the food base is cooked to develop flavors and integrate ingredients. The food base can reduce, creating a thicker consistency and lower water activity, which contributes to shelf stability without artificial preservatives. Also during cooking, the evaporation may be controlled. The evaporation may be controlled to achieve a desired thickness and reduce free water content, making the sauce less susceptible to microbial growth while preserving a rich texture.

In particular embodiments, components of the food mixture can be encapsulated. In some cases, the preparation of the composition includes encapsulation of select ingredient (e.g., probiotics). In particular embodiments, encapsulation includes microencapsulation. Microencapsulation can provide a protective layer for the encapsulated ingredients, enhancing stability by shielding ingredients from the acidic environment and any mild heat that may be present during the preparation. In embodiments that microencapsulate ingredients, the microencapsulated ingredients can be stored in a controlled environment until ready for use to maintain their potency and viability. Encapsulation can enhance the stability of the ingredients. Encapsulation can be accomplished by methods known to those skilled in the art. For example, encapsulation includes trapping the material of interest within a matrix. The choice of matrix material will depend on desired characteristics for release, dispersion, stability, and rheology. Example matrix materials include maltodextrin, pectin, gum, cyclodextrins, carboxymethyl, whey protein isolates, and soy protein isolates. Encapsulation can be accomplished by spray drying, extrusion, coacervation, spray-chilling/cooling, freeze-drying, and ionic gelation.

The addition of sensitive ingredients (e.g., probiotics) to a composition can include cooling the composition to safe temperatures and incorporating the sensitive ingredients when the composition is at the target temperature. After cooking, the food product is cooled to 100-115° F. Once the target temperature is reached the sensitive ingredients can be added. In particular embodiments, the added sensitive ingredients are stirred and/or distributed evenly throughout the composition.

In particular embodiments, the food product mixture is packaged in dark glass jars to block harmful UV and visible light, which can degrade sensitive compounds. This packaging solution preserves ingredient potency and stability. The use of optional encapsulation, dark glass, and an acidic base provides multi-layered protection for the active ingredients, ensuring they remain potent and bioavailable.

In particular embodiments, components to the food mixture are hydrolyzed. In particular embodiments, components of the protein mixture are hydrolyzed. The protein may be hydrolyzed enzymatically or chemically. For enzymatic hydrolysis, specific enzymes (e.g., proteases) can be used to cleave peptide bonds at specific sites within the protein. Example enzymes include plant proteases (e.g., bromelain, papain), animal proteases (e.g., pancreatin, trypsin, chymotrypsin), and bacterial proteases (e.g., pronase E, alcalase). In particular embodiments, the enzyme includes trypsin, chymotrypsin, pepsin, alkaline protease, papain, bromelain, alcalase, thermolysin, or flavourzyme Hydrolysis may be controlled, for example, through temperature, time, pH, and enzyme/substrate ration. Chemical hydrolysis uses acids or bases to break down the peptide bonds within a protein. Acid hydrolysis is often used with hydrochloric acid at high temperatures. Alkaline hydrolysis uses strong bases like sodium hydroxide and potassium hydroxide. The hydrolyzed protein may be completely hydrolyzed, partially hydrolyzed, or may be a combination of intact protein (i.e., not hydrolyzed) and hydrolyzed protein (i.e., completely or partially hydrolyzed protein).

Collagen is a tough, triple-helix protein rich in glycine, proline, and hydroxyproline. In particular embodiments, collagen can be broken down using enzymes like pepsin (acidic conditions), papain, bromelain, and alcalase. These enzymes release smaller peptides for improved solubility, digestibility, and bioavailability. Bone broth protein is derived from animal bones rich in collagen and gelatin. In particular embodiments, bone broth protein can be hydrolyzed with enzymes like alcalase (efficient collagen breakdown), papain, and flavourzyme to release bioactive peptides with health benefits, such as joint support and gut health. In particular embodiments, whey proteins, including beta-lactoglobulin and alpha-lactalbumin, are hydrolyzed with enzymes like trypsin, chymotrypsin, and alcalase to reduce allergenicity, enhance digestibility, and create bioactive peptides.

The degree of hydrolysis is the extent to which peptide bonds are broken by a hydrolysis chemical or enzymatic reaction. To quantify the partially hydrolyzed protein component of a protein mixture, the degree of protein hydrolysis can be determined by quantifying the amino nitrogen to total nitrogen ratio (AN/TN) of the protein component of the selected nutritional formula. The amino nitrogen component is quantified by USP titration methods for determining amino nitrogen content, while the total nitrogen component is determined by the Tecator® Kjeldahl method. The soluble protein concentration and the degree of hydrolysis may be measured by any suitable method, including a chromatographic method (as described in Food. Chem., 125 (2011) 1041-1050).

In particular embodiments, the method further includes adjusting the pH of the admixture to a pH in the range of 3.0-4.8. In particular embodiments, the method further includes packaging the admixture. In particular embodiments, the packaging further includes heat-treating the admixture. While not always being necessary, in particular embodiments, the admixture is subjected to homogenization. In particular embodiments, the food base is subjected to homogenization before it is mixed with the collagen peptide, whey protein, bone broth protein, and/or adaptogens. It has been determined that a shelf-stable product that does not require refrigeration or does not undergo sufficient heat treatment must have a pH equal to or below 4.6. In particular embodiments, the product described herein has a pH in the range of 3.0-4.8. A method of adjusting the pH includes adding an edible acid to the food product, admixture, or components within the food product (e.g., food base, adaptogens, collagen peptide, whey protein, and/or bone broth protein). In particular embodiments, the amount of edible acid that needs to be added to the food product will be dependent on the acidity of the food product. For example, if the food product includes tomatoes, the acidity of the tomatoes will affect the pH of the food product and the amount of edible acid to reach the desired pH range.

Methods of measuring pH include measuring pH using an indicator (liquid or test paper), hydrogen-electrode, quinhydrone-electrode, antimony-electrode, glass electrode, or a semiconductor sensor. In particular embodiments, a hydrogen-electrode is used to measure pH. A hydrogen-electrode is made by adding platinum black to platinum wire or a platinum plate. It is immersed in the test solution (e.g., food product) and an electric charge is applied to the solution and the solution is saturated with hydrogen gas. The electrode potential is measured between the platinum black electrode and silver chloride electrode and the measured potential is inversely proportional to pH of the solution.

The composition can be preserved and packaged. Any of the conventional packaging processes known for the manufacture of shelf-stable products can be employed herein, such as retorting, high-temperature-short-time sterilization, or hot-fill-hold processing. The various processing parameters are adjusted to achieve a commercially sterile shelf-stable product.

In particular embodiments, packaging includes cold-filled packaging and sealing. In particular embodiments, the composition is cold-filled into a sterile container under sterile conditions. In particular embodiments, the composition is transferred to the container at or below room temperature. In particular embodiments, heat exposure is avoided to prevent sensitive ingredient (e.g., probiotic) degradation. In particular embodiments, sealing the container includes removing oxygen from the container before sealing. This can prevent aerobic bacterial growth and oxidation. Sealing the container includes vacuum sealing or nitrogen flushing. In particular embodiments, the container supports natural preservation methods and sensitive ingredient (e.g., probiotic) stability. In particular embodiments, the container is a glass jar. In particular embodiments, the container is a dark glass jar.

In other embodiments, the mixture is subjected to high-temperature-short-time processing for cooking and commercial sterilization. High-temperature-short-time processing was developed to provide a means of destroying acid-tolerant, heat-resistant spoilage organisms which could not be destroyed by conventional processes without a severe loss in the flavor and quality of the product. The process relies upon two principles: First, the increase in rate of heat penetration affected in high-efficiency heat exchangers decreases the processing time needed to heat the product to a given temperature. Second, the use of a high processing temperature for a short time reduces the degree of chemical change in the product for a given sterilization value.

Coil, plate, and tubular heat exchangers have all been used in high-temperature-short-time processing. Certain conditions should be considered in judging the merits of a particular type of equipment, such as the thermal efficiency, ease of cleaning of the heat exchanger surfaces, and available means of automatic control of times and temperatures.

The heat exchanger must be designed to withstand the pressures necessary to pump the required amount of product through the equipment, as well as being capable of heating the product to the sterilizing temperature. The pressure throughout the heat exchanger should exceed the pressure of saturated steam at the maximum temperature in the heater, to avoid boiling within the heat exchanger, which reduces heat transfer efficiency. The pump used must maintain a positive constant flow rate against the pressures necessary to force the product through the heat exchanger. This is especially important because flow rate through the heat exchanger will determine the time-temperature exposure of the product. In particular embodiments, the high-temperature-short-time equipment is operated under automated control to maintain the desired commercial sterilizing times and temperatures.

Any high-temperature-short-time system can be used. In practice, the high-temperature-short-time method includes heating the product rapidly and continuously to a temperature of from 220° F. (104° C.) to 280° F. (137° C.) in a heat exchanger for a time appropriate to achieve sterilization. In general, 5 seconds to 60 seconds time is sufficient at temperatures from 245° F. (118° C.) to 280° F. (138° C.) to commercially sterilize the product. The product is then rapidly cooled by flashing the steam off through release of pressure or by passing the product through a heat exchanger at lower temperatures. The product is cooled in this manner to a temperature of from 140° F. (60° C.) to 160° F. (71° C.).

Heat treating the product before packaging can be performed. In particular embodiments, heat-treating includes heating the product to at least 80° C. for at least 1 minute, at least 80° C. for at least 5 minutes, or at least 85° C. for at least 10 minutes. As will be appreciated by the skilled person, even higher temperatures and longer exposure times may be used. In particular embodiments, the food product is heated at a temperature of 200° F. for 10 mins.

The duration of heating depends on the temperature(s) of heating. Typically, at higher temperatures, a lower duration of heating can be necessary compared to lower temperatures. Nevertheless, in particular embodiments, the heat-treatment must be sufficient to lower the microbial load such that the product has a prolonged shelf-life when stored at ambient temperatures (e.g., in the range of 70-150° C.) and maintaining the temperature in that range for a duration sufficient to kill a substantial number of the viable microorganisms. In particular embodiments, the food product shelf-life includes at least 1 month, at least 2 months, at least 3 months, at least 4 months, at least 5 months, at least 6 months, at least 7 months, at least 8 months, at least 9 months, at least 10 months, at least 11 months, or over a year.

Optionally, the product is packed in hermetically sealed containers. In particular embodiments, the product is packed in hermetically sealed containers using a hot-fill-hold technique. While “hermetically sealed containers” is usually used to refer to jars and cans, it can be appreciated that other aseptic packages, such as plastic and foil plastic laminate pouches, can also be adapted to this process. In hot-fill-hold processing the container is filled with product at a temperature of at least 180° F. (82° C.) and held for 2 to 5 minutes before cooling. In the hot-fill-hold process it is extremely important to assure this high closing temperature for each and every container. Without a high closing temperature, the temperature of the product prior to closing may drop below temperatures which are effective to pasteurize any vegetative bacteria, yeast and molds on the inner surfaces of the container. Therefore, time between container filling and container sealing is minimized. In the event the process is stopped, such as by equipment breakdown, containers in transit between filling and sealing operations will probably cool excessively and will need to be reprocessed, reheated or scraped. In particular embodiments, the packaging in a sealed container includes using an inert atmosphere to pressurize the product in the sealed container. In particular embodiments, the container is filled with product at a temperature of 200° F.

After the container is sealed it is important that portions of the container not contacted by product be heated to the same temperature as the product. For example, if jars are filled with product, a post-capping hot water spray directed at the lid and headspace of the jar can be used to provide for lid and headspace heating. Alternatively, the jars can be inverted after filling and capping for the desired hold time to provide for lid and headspace sterilization. The product is then cooled to storage temperatures. In particular embodiments, storage temperatures include room temperature or colder. In particular embodiments, storage temperatures include 80° F. (26.7° C.) or less, 75° F. (23.9° C.) or less, 70° F. (21.1° C.) or less, 65° F. (18.3° C.) or less, 60° F. (15.6° C.) or less, 55° F. (12.8° C.) or less, 50° F. (10° C.) or less, 45° F. (7.2° C.) or less, 40° F. (4.4° C.) or less, 35° F. (1.7° C.) or less, or 30° F. (−1.1° C.) or less.

It can be appreciated that still other embodiments can be devised without departing from its scope and spirit and without losing its advantages. Minor processing parameters and product ingredients can be altered without departing from the scope of the disclosure.

The products within the scope of the disclosure may take a variety of forms. For instance, the product may be manufactured and sold as a ready-to-eat food product for direct consumption by a subject or a ready-to-cook food product. In other embodiments, the product may be prepared in concentrate or powder form to be reconstituted for use by the subject by the addition of water, other liquid, or food product. Such reconstitution is made with the requisite amounts of water/liquid/food product to ensure that the product to be consumed contains the active components in the proportions previously noted. Alternatively, the composition described herein can be prepared as a dry powder mixture. In particular embodiments, the dry powder mixture can be added to water or other food products for reconstitution, thereby creating an adaptogenic, high-protein food product. In other examples, the powder mixture can be added to a suitable support for tablet compression.

In particular embodiments, additional ingredients (e.g., spices, sugar, salt, meat) can be added to the composition disclosed herein. In particular embodiments, the composition disclosed herein can be used as an ingredient in other products.

Quality control and stability testing methods are known to those skilled in the art. In particular embodiments, quality control and stability testing includes pH testing, consistency testing, probiotic viability testing, and shelf-life monitoring. Particular embodiments include verifying that the composition maintains a pH of 4.6 or below. Particular embodiments include ensuring that the composition remains shelf-stable and safe for consumption. Particular embodiments include testing that the live probiotic counts meets the intended health benefits. Particular embodiments include shelf stability testing in a temperature-controlled environment to confirm that the composition or food product remains stable at room temperature while maintaining probiotic viability.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (IX) EXEMPLARY EMBODIMENTS

1. An adaptogenic, high-protein (AHP) sauce including:
   a protein mixture at a concentration by weight of 1%-25%,
   adaptogens at a concentration by weight of 0.01%-7%,
   a food base at a concentration by weight of 50%-99%, and
   an edible acid at a concentration by weight of 0%-2%,
   wherein the concentrations do not exceed 100%.
2. The AHP sauce of embodiment 1, wherein the adaptogens include a mushrooms, holy basil (Tulsi), ashwaganda, Rhodiola Rosea, curcumin, ginseng, Shcisandra, Astragalus, Goji berry, Moringa, Licorice root, Dangshen, Eleuthero, or Jiaogulan.
3. The AHP sauce of embodiments 1 or 2, wherein the adaptogens include mushrooms, holy basil, ashwaganda, Rhodiola Rosea, or curcumin.
4. The AHP sauce of any of embodiments 1-3, wherein the adaptogens include mushrooms, holy basil, ashwaganda, Rhodiola Rosea, and curcumin.
5. The AHP sauce of any of embodiments 2-4, wherein the mushrooms include Reishi, Lion's mane, cordyceps, Chaga, Turkey tail, Maitake, and/or Shiitake.
6. The AHP sauce of any of embodiments 2-5, wherein the mushrooms include Reishi, Lion's mane, cordyceps, Chaga, or Turkey tail.
7. The AHP sauce of any of embodiments 2-6, wherein the mushrooms include Reishi, Lion's mane, cordyceps, Chaga, and/or Turkey tail.
8. The AHP sauce of any of embodiments 2-7, wherein the curcumin includes turmeric.
9. The AHP sauce of any of embodiments 1-8, wherein the protein mixture includes a collagen peptide, a whey protein, and/or a bone broth protein.
10. The AHP sauce of any of embodiments 1-9, wherein the AHP sauce includes a pH of between 3.0 and 4.8.
11. The AHP sauce of any of embodiments 1-10, further including a taste-masking agent.
12. The AHP sauce of embodiment 11, wherein the taste-masking agent includes a bitter blocker.
13. The AHP sauce of any of embodiments 1-12, further including a fiber mixture.
14. The AHP sauce of embodiment 13, wherein the fiber mixture includes a soluble fiber and/or an insoluble fiber.
15. The AHP sauce of embodiments 13 or 14, wherein the fiber mixture includes a soluble fiber and an insoluble fiber.
16. The AHP sauce of embodiment 15, wherein the soluble fiber is derived from chicory root, tapioca, oats, peas, beans, apples, bananas, avocados, citrus fruits, carrots, barley, or psyllium.
17. The AHP sauce of embodiments 15 or 16, wherein the soluble fiber includes chicory root or tapioca fiber.
18. The AHP sauce of any of embodiments 15-17, wherein the insoluble fiber is derived from vegetables or grains.
19. The AHP sauce of any of embodiments 15-18, wherein the insoluble fiber is derived from seeds, nuts, dark green leafy vegetables, or wheat bran.
20. The AHP sauce of any of embodiments 1-19, further including probiotics.
21. The AHP sauce of embodiment 20, wherein the probiotics include a Lactobacillus or a Bifidobacterium.
22. The AHP sauce of embodiment 21, wherein the Lactobacillus includes Lactobacillus acidophilus, Lactobacillus bifidus, Lactobacillus brevis, Lactobacillus bulgaricus, Lactobacillus delbrucki, Lactobacillus casei, Lactobacillus cellobiosus, Lactobacillus fermentum, Lactobacillus gasseri, Lactobacillus germentum, Lactobacillus helveticus, Lactobacillus johnsonhii, Lactobacillus lactis, Lactobacillus leichimanii, Lactobacillus plantarum, Lactobacillus reuteri, Lactobacillus rhamnosus, Lactobacillus sake, Lactobacillus salivaroes, Lactobacillus thermophilus, or Lactobacillus xylosus.
23. The AHP of embodiments 21 or 22, wherein the Bifidobacterium includes Bifidobacterium adolescentis, Bifidobacterium bifidum, Bifidobacterium breve, Bifidobacterium cereus, Bifidobacterium infantis, Bifidobacterium lactis, Bifidobacterium longum, or Bifidobacterium thermophilus.
24. The AHP sauce of any of embodiments 9-23, wherein the collagen peptide includes a protein grade of C, C+, B, B+, A, A+, or A++.
25. The AHP sauce of any of embodiments 9-24, wherein the collagen peptide includes a protein grade of A, A+, or A++.
26. The AHP sauce of any of embodiments 9-25, wherein the whey protein includes a protein grade of C, C+, B, B+, A, A+, or A++.
27. The AHP sauce of any of embodiments 9-26, wherein the whey protein includes a protein grade of A, A+, or A++.
28. The AHP sauce of any of embodiments 9-27, wherein the bone broth protein includes a protein grade of C, C+, B, B+, A, A+, or A++.
29. The AHP sauce of any of embodiments 9-28, wherein the bone broth protein includes a protein grade of A, A+, or A++.
30. The AHP sauce of any of embodiments 1-29, wherein the AHP sauce does not include synthetic ingredients.
31. The AHP sauce of any of embodiments 1-30, wherein the food base includes a pasta sauce.
32. The AHP sauce of any of embodiments 1-31, wherein the AHP sauce does not include added sugar.
33. A composition including:
   a protein mixture at a concentration by weight of 1-25%,
   adaptogens at a concentration by weight of 0.01-7%,
   a food base at a concentration by weight of 50-99%, and
   an edible acid at a concentration by weight of 0-2%;
   wherein the concentrations do not exceed 100%.
34. The composition of embodiment 33, wherein the adaptogens include a mushrooms, holy basil (Tulsi), ashwaganda, Rhodiola Rosea, curcumin, ginseng, Shcisandra, Astragalus, Goji berry, Moringa, Licorice root, Dangshen, Eleuthero, or Jiaogulan.
35. The composition of embodiments 33 or 34, wherein the adaptogens include mushrooms, holy basil, ashwaganda, Rhodiola Rosea, or curcumin.
36. The composition of any of embodiments 33-35, wherein the adaptogens include mushrooms, holy basil, ashwaganda, Rhodiola Rosea, and curcumin.
37. The composition of any of embodiments 34-36, wherein the mushrooms include Reishi, Lion's mane, cordyceps, Chaga, Turkey tail, Maitake, and/or Shiitake.
38. The composition of any of embodiments 34-37, wherein the mushrooms include Reishi, Lion's mane, cordyceps, Chaga, or Turkey tail.
39. The composition of any of embodiments 34-38, wherein the mushrooms include Reishi, Lion's mane, cordyceps, Chaga, and/or Turkey tail.
40. The composition of any of embodiments 34-39, wherein the curcumin includes turmeric.
41. The composition of any of embodiments 33-40, wherein the protein mixture includes a collagen peptide, a whey protein, and/or a bone broth protein.
42. The composition of any of embodiments 33-41, wherein the protein mixture includes:
   a collagen peptide at a concentration by weight of 0-25%,
   a whey protein at a concentration by weight of 0-20%, and
   a bone broth protein at a concentration by weight of 0-20%,
   wherein the concentrations do not exceed 25% of the composition.
43. The composition of any of embodiments 33-42, wherein the protein mixture includes hydrolyzed and/or non-hydrolyzed proteins.
44. The composition of any of embodiments 41-5143 wherein the collagen peptide includes at least 0.5 g, at least 0.6 g, at least 0.7 g, at least 0.8 g, at least 0.81 g, or at least 0.9 g of protein per gram of collagen peptide.
45. The composition of any of embodiments 41-44, wherein the whey protein includes at least 0.5 g, at least 0.6 g, at least 0.7 g, at least 0.8 g, at least 0.85 g, at least 0.86 g, or at least 0.9 g of protein per gram of whey protein.
46. The composition of any of embodiments 41-45, wherein the bone broth protein includes at least 0.5 g, at least 0.6 g, at least 0.7 g, at least 0.8 g, at least 0.81 g, or at least 0.9 g of protein per gram of bone broth protein.
47. The composition of any of embodiments 33-46, wherein the food base includes pasta sauce, taco sauce, barbecue sauce, soup base, chili sauce, hot sauce, curry, masala, chutney, or salsa.
48. The composition of any of embodiments 33-47, wherein the edible acid is selected from the group including citric acid, malic acid, tartaric acid, acetic acid, benzoic acid, butyric acid, lactic acid, fumaric acid, succinic acid, ascorbic acid, adipic acid, phosphoric acid, and mixtures thereof.
49. The composition of any of embodiments 33-48, wherein the edible acid includes citric acid.
50. The composition of any of embodiments 33-49, wherein the composition includes a pH of between 3.0 and 4.8.
51. The composition of any of embodiments 33-50, wherein the composition includes a total protein content of at least 1%, at least 2%, or at least 2.5%.
52. The composition of any of embodiments 33-51, further including a taste-masking agent.
53. The composition of embodiment 52, wherein the taste-masking agent includes a bitter blocker.
54. The composition of embodiment 53, wherein the bitter blocker is at a concentration by weight of less than 1%, less than 0.1%, less than 0.01%, or less than 0.001%.
55. The composition of any of embodiments 33-54, further including a fiber mixture.
56. The composition of embodiment 55, wherein the fiber mixture includes a soluble fiber and/or an insoluble fiber.
57. The composition of embodiments 55 or 56, wherein the fiber mixture includes a soluble fiber and an insoluble fiber.
58. The composition of embodiments 56 or 57, wherein the soluble fiber is derived from chicory root, tapioca, oats, peas, beans, apples, bananas, avocados, citrus fruits, carrots, barley, or psyllium.
59. The composition of any of embodiments 56-58, wherein the soluble fiber includes chicory root.
60. The composition of any of embodiments 56-59, wherein the soluble fiber includes tapioca fiber.
61. The composition of any of embodiments 56-60, wherein the insoluble fiber is derived from vegetables or grains.
62. The composition of any of embodiments 56-61, wherein the insoluble fiber is derived from seeds, corn, nuts, dark green leafy vegetables, or wheat bran.
63. The composition of any of embodiments 33-62, further including probiotics.
64. The composition of embodiment 63, wherein the probiotics include a Lactobacillus or a Bifidobacterium.
65. The composition of embodiment 64, wherein the Lactobacillus includes Lactobacillus acidophilus, Lactobacillus bifidus, Lactobacillus brevis, Lactobacillus bulgaricus, Lactobacillus delbrucki, Lactobacillus casei, Lactobacillus cellobiosus, Lactobacillus fermentum, Lactobacillus gasseri, Lactobacillus germentum, Lactobacillus helveticus, Lactobacillus johnsonhii, Lactobacillus lactis, Lactobacillus leichimanii, Lactobacillus plantarum, Lactobacillus reuteri, Lactobacillus rhamnosus, Lactobacillus sake, Lactobacillus salivaroes, Lactobacillus thermophilus, or Lactobacillus xylosus.
66. The composition of embodiments 64 or 65, wherein the Bifidobacterium includes Bifidobacterium adolescentis, Bifidobacterium bifidum, Bifidobacterium breve, Bifidobacterium cereus, Bifidobacterium infantis, Bifidobacterium lactis, Bifidobacterium longum, or Bifidobacterium thermophilus.
67. The composition of any of embodiments 63-66, wherein the probiotics are at a concentration by weight of 0.05-3% of the composition.
68. The composition of any of embodiments 63-67, wherein the probiotics are at a concentration by weight of 0.1-1% of the composition.
69. The composition of any of embodiments 33-68, wherein the protein mixture includes a protein grade of C, C+, B, B+, A, A+, or A++.
70. The composition of any of embodiments 33-69, wherein the protein mixture includes a protein grade of A, A+, or A++.
71. The composition of any of embodiments 41-70, wherein the collagen peptide includes a protein grade of C, C+, B, B+, A, A+, or A++.
72. The composition of any of embodiments 41-71, wherein the collagen peptide includes a protein grade of A, A+, or A++.
73. The composition of any of embodiments 41-72, wherein the whey protein includes a protein grade of C, C+, B, B+, A, A+, or A++.
74. The composition of any of embodiments 41-73, wherein the whey protein includes a protein grade of A, A+, or A++.
75. The composition of any of embodiments 41-74, wherein the bone broth protein includes a protein grade of C, C+, B, B+, A, A+, or A++.
76. The composition of any of embodiments 41-75, wherein the bone broth protein includes a protein grade of A, A+, or A++.
77. The composition of any of embodiments 33-76, wherein the composition does not include synthetic ingredients.
78. The composition of any of embodiments 33-77, wherein the composition does not include added sugar.
79. A method of producing an adaptogenic high-protein (AHP) sauce including:
   forming a composition including:
   a) 1-25 weight % of a protein mixture,
   b) 0.01-7 weight % of adaptogens, and
   c) 50-99 weight % of a food base, wherein a summation of weight % s does not exceed 100%;
   adjusting a pH of the AHP sauce; and
   packaging the composition.
80. The method of embodiment 79, wherein the adaptogens include a mushrooms, holy basil (Tulsi), ashwaganda, Rhodiola Rosea, curcumin, ginseng, Shcisandra, Astragalus, Goji berry, Moringa, Licorice root, Dangshen, Eleuthero, or Jiaogulan.
81. The method of embodiments 79 or 80, wherein the adaptogens include mushrooms, holy basil, ashwaganda, Rhodiola Rosea, or curcumin.
82. The method of any of embodiments 79-81, wherein the adaptogens include mushrooms, holy basil, ashwaganda, Rhodiola Rosea, and curcumin.
83. The method of any of embodiments 80-82, wherein the mushrooms include Reishi, Lion's mane, cordyceps, Chaga, Turkey tail, Maitake, and/or Shiitake.
84. The method of any of embodiments 80-83, wherein the mushrooms include Reishi, Lion's mane, cordyceps, Chaga, or Turkey tail.
85. The method of any of embodiments 80-84, wherein the mushrooms include Reishi, Lion's mane, cordyceps, Chaga, and/or Turkey tail.
86. The method of any of embodiments 80-85, wherein the curcumin includes turmeric.
87. The method of any of embodiments 79-86, wherein the adjusting the pH includes adjusting the pH to less than 4.8.
88. The method of any of embodiments 79-87, wherein the adjusting the pH includes adjusting the pH to less than 4.6.
89. The method of any of embodiments 79-88, wherein the adjusting the pH includes adding 0-2 weight % of an edible acid.
90. The method of any of embodiments 79-89, wherein the protein mixture includes a collagen peptide, a whey protein, and/or a bone broth protein.
91. The method of any of embodiments 79-90, wherein the protein mixture includes:
   a) 0-20 weight % of a collagen peptide of the composition,
   b) 0-20 weight % of a whey protein of the composition, and
   c) 0-20 weight % of a bone broth protein of the composition;
   wherein a summation of weight % s does not exceed 25% of the composition.
92. The method of any of embodiments 79-91, wherein the forming the composition includes cooking the composition.
93. The method of embodiment 92, wherein the cooking the composition includes staged cooking.
94. The method of embodiment 93, wherein the staged cooking includes in order:
   cooking early-stage ingredients;
   cooking mid-stage ingredients; and
   cooking late-stage ingredients.
95. The method of embodiment 94, wherein the early-stage ingredients include Reishi, Lion's mane, cordyceps, Chaga, Turkey tail, holy basil, ashwaganda, Rhodiola Rosea, curcumin, protein mixture, and/or food base.
96. The method of embodiments 94 or 95, wherein the early-stage ingredients include curcumin, chaga, and/or reishi.
97. The method of any of embodiments 94-96, wherein the mid-stage ingredients include Reishi, Lion's mane, cordyceps, Chaga, Turkey tail, holy basil, ashwaganda, Rhodiola Rosea, curcumin, protein mixture, and/or food base.
98. The method of any of embodiments 94-97, wherein the mid-stage ingredients include cordyceps and/or ashwagandha.
99. The method of any of embodiments 94-98, wherein the late-stage ingredients include Reishi, Lion's mane, cordyceps, Chaga, Turkey tail, holy basil, ashwaganda, Rhodiola Rosea, curcumin, protein mixture, and/or food base.
100. The method of any of embodiments 94-99, wherein the late-stage ingredients include holy basil, Rhodiola Rosea, Lion's mane, and/or Turkey tail.
101. The method of any of embodiments 79-100, wherein packaging the composition includes cold-filled packaging and sealing.
102. The method of any of embodiments 79-101, wherein packaging the composition includes heat treating the composition prior to, during, or after packaging.
103. The method of any of embodiments 79-102, wherein the composition includes one or more heat-treated ingredients.
104. The method of any of embodiments 79-103, wherein the AHP sauce includes a total protein content of at least 1%, at least 2%, or at least 2.5%.
105. The method of any of embodiments 79-104, wherein the protein mixture includes a protein grade of C, C+, B, B+, A, A+, or A++.
106. The method of any of embodiments 79-105, wherein the protein mixture includes a protein grade of A, A+, or A++.
107. The method of any of embodiments 90-106, wherein the collagen peptide includes a protein grade of C, C+, B, B+, A, A+, or A++.
108. The method of any of embodiments 90-107, wherein the collagen peptide includes a protein grade of A, A+, or A++.
109. The method of any of embodiments 90-108, wherein the whey protein includes a protein grade of C, C+, B, B+, A, A+, or A++.
110. The method of any of embodiments 90-109, wherein the whey protein includes a protein grade of A, A+, or A++.
111. The method of any of embodiments 90-110, wherein the bone broth protein includes a protein grade of C, C+, B, B+, A, A+, or A++.
112. The method of any of embodiments 90-111, wherein the bone broth protein includes a protein grade of A, A+, or A++.
113. The method of any of embodiments 79-112, wherein the protein mixture includes a hydrolyzed protein and/or a non-hydrolyzed protein.
114. The method of any of embodiments 79-113, further including hydrolyzing at least part of the protein mixture.
115. The method of embodiment 114, wherein the hydrolyzing includes providing an enzyme or a chemical to the protein mixture.
116. The method of embodiment 115, wherein the enzyme includes a plant enzyme, an animal enzyme, or a bacterial enzyme.
117. The method of embodiments 115 or 116, wherein the enzyme includes trypsin, chymotrypsin, pepsin, alkaline protease, papain, bromelain, alcalase, thermolysin, or flavourzyme.
118. The method of any of embodiments 115-117, wherein the chemical includes an acid or base.
119. The method of any of embodiments 79-118, wherein the composition further includes a taste-masking agent.
120. The method of embodiment 119, wherein the taste-masking agent includes a bitter blocker.
121. The method of any of embodiments 79-120, wherein the composition further includes a fiber mixture.
122. The method of embodiment 121, wherein the fiber mixture includes a soluble fiber and/or an insoluble fiber.
123. The method of embodiments 121 or 122, wherein the fiber mixture includes a soluble fiber and an insoluble fiber.
124. The method of embodiments 122 or 123, wherein the soluble fiber is derived from chicory root, tapioca, corn, oats, peas, beans, apples, bananas, avocados, citrus fruits, carrots, barley, or psyllium.
125. The method of any of embodiments 122-124, wherein the soluble fiber includes chicory root.
126. The method of any of embodiments 122-125, wherein the soluble fiber includes tapioca fiber.
127. The method of any of embodiments 122-126, wherein the soluble fiber includes a concentration by weight of 0-10%, 3-8%, 4-6%, or 4.8-5.6%.
128. The method of any of embodiments 122-127, wherein the insoluble fiber is derived from vegetables or grains.
129. The method of any of embodiments 122-128, wherein the insoluble fiber is derived from corn, seeds, nuts, dark green leafy vegetables, or wheat bran.
130. The method of any of embodiments 122-129, wherein the insoluble fiber includes a concentration by weight of 0-10%, 0-8%, 0-6%, or 0-1%.
131. The method of any of embodiments 79-130, further including probiotics.
132. The method of embodiment 131, wherein the probiotics include a Lactobacillus and/or a Bifidobacterium.
133. The method of embodiment 132, wherein the Lactobacillus includes Lactobacillus acidophilus, Lactobacillus bifidus, Lactobacillus brevis, Lactobacillus bulgaricus, Lactobacillus delbrucki, Lactobacillus casei, Lactobacillus cellobiosus, Lactobacillus fermentum, Lactobacillus gasseri, Lactobacillus germentum, Lactobacillus helveticus, Lactobacillus johnsonhii, Lactobacillus lactis, Lactobacillus leichimanii, Lactobacillus plantarum, Lactobacillus reuteri, Lactobacillus rhamnosus, Lactobacillus sake, Lactobacillus salivaroes, Lactobacillus thermophilus, or Lactobacillus xylosus.
134. The method of embodiments 132 or 133, wherein the Bifidobacterium includes Bifidobacterium adolescentis, Bifidobacterium bifidum, Bifidobacterium breve, Bifidobacterium cereus, Bifidobacterium infantis, Bifidobacterium lactis, Bifidobacterium longum, or Bifidobacterium thermophilus.
135. The method of any of embodiments 131-134, further including encapsulating the probiotics before forming the composition.
136. The method of any of embodiments 79-135, wherein the composition does not include synthetic ingredients.
137. The method of any of embodiments 79-136, wherein the composition does not include added sugar.

### (X) EXAMPLES

### Example 1. A High Protein Pasta Sauce was Prepared According to the Present Disclosure

The particular ingredients and the amounts of each ingredient included in the high protein pasta sauce are listed below in Table 1. The protein was provided by a blend of collagen peptide, bone broth protein, and whey protein (specifically whey isolate). The edible acid used to adjust the pH is citric acid. The high protein pasta sauce composition was shelf-stable and had a flavor that does not significantly vary from the food base, pasta sauce.

TABLE 1
Formulation for a high protein pasta sauce.

| | Pasta | | Collagen | | Bone Broth | | Whey | | Citric | |
|---|---|---|---|---|---|---|---|---|---|---|
| | Sauce | | Peptide | | Protein | | Isolate | | Acid | |
| Protein per gram of ingredient | 0.016 | g | 0.81 | g | 0.81 | g | 0.86 | g | 0 | g |
| Mass per serving | 125 | g | 15 | g | 7 | g | 1 | g | 0.8 | g |
| Protein Content | 2 | g | 12.15 | g | 5.67 | g | 0.86 | g | 0 | g |
| Fat Content | 3.5 | g | 0 | g | 0 | g | 0 | g | 0 | g |

### Example 2. A High Protein Pasta Sauce was Prepared According to the Present Disclosure

The particular ingredients and the amounts of each ingredient included in the high protein pasta sauce are listed below in Table 2. The collagen (collagen peptide), broth (bone broth), and whey protein were mixed together and whisked into the water. Once all ingredients were mixed together, the pH of the mixture was tested. Additional citric acid was added to further reduce the pH of the mixture before packaging. Herein a total of 1 weight % of citric acid was included in the mixture. To begin the packaging procedure, the mixture was heated to 200° F. for 10 minutes. The mixture was packaged into 24 oz pasta sauce jars (63 twist, R-1121) with a lid (63-2030 black metal lug, but top (Siligan) 1300/cs). During packaging, the fill temperature was set to 180° F. with a minimum fill temperature of 165° F. for 1 minute. The cap was sterilized by steam or inversion.

TABLE 2
Formulation for a high protein pasta sauce.

| | Prod. | Portion |
|---|---|---|
| Item Description | Wt. | (%) |
| Basil leaf (whole, dried, imported) | 2.13 | 0.036 |
| Garlic (dehydrated, minced, C-50-GM) | 3.41 | 0.057 |
| Garlic (powder, C-50-GP) | 0.85 | 0.014 |
| Oil (e.g., olive oil or canola oil) | 119.45 | 1.991 |
| Oil, Extra Virgin Olive (Piano Del Principe) | 1.28 | 0.021 |
| Onion, Yellow (IQF, rstd, ⅜ dice, C-31-OYTD) | 106.65 | 1.777 |
| Onion, (dehydrated, minced) | 10.66 | 0.178 |
| Oregano Leaves, Mediterranean (dried, whole) | 0.85 | 0.014 |
| Parsley (dried flakes) (22# tin) | 2.13 | 0.036 |
| Pepper, Black (28 mesh) | 0.85 | 0.014 |
| Salt, Sea (untreated, Cargill) | 78.92 | 1.315 |
| Tomatoes (diced in juice, ¾″) (Drum) | 665.48 | 11.091 |
| Tomato, Paste (31% Hot Break, .060 screen) | 1164.59 | 19.410 |
| Water | 3082.55 | 51.376 |
| Carrots (shredded, fresh, 1/16″) | 92.57 | 1.543 |
| Collagen | 426.59 | 7.110 |
| Broth | 204.76 | 3.413 |
| Whey | 29.86 | 0.498 |
| Citric Acid (anhydrous, fine) | 6.40 | 0.107 |
| Total Test Batch Weight | 6,000 | grams |

### Example 3. Synergies of Adaptogens with Tomato Sauce Base

A tomato food base offers unique synergistic benefits that enhance the adaptogenic properties and overall stability of the food product (e.g., sauce).

Enhanced Bioavailability and Absorption of Key Compounds. The tomato's natural acidity enhances the absorption and bioavailability of fat-soluble and heat-sensitive compounds, such as curcumin and withanolides. This ensures maximum effectiveness of each adaptogenic ingredient, supporting both metabolic and immune functions. Lycopene's antioxidant properties, combined with adaptogenic mushrooms like chaga and reishi, create a powerful cell-protective effect that helps reduce oxidative stress.

Stabilization and Preservation of Sensitive Compounds. The acidic tomato base acts as a natural preservative, extending the shelf life of heat-sensitive compounds and preventing oxidation. This reduces the need for artificial preservatives, aligning with consumer preferences for clean-label products. The tomato base's stabilizing effect minimizes the need for encapsulation, making the product simpler and more cost-effective to produce while maintaining ingredient efficacy.

Balanced Flavor Profile and Masking of Bitter Adaptogens. The tomato's acidity and natural umami help to mask the earthy flavors of adaptogens like ashwagandha and Rhodiola Rosea, creating a balanced flavor profile. The acidity of tomatoes heightens the aromatic properties of spices like ginger and holy basil, creating a well-rounded taste without artificial flavor enhancers.

Anti-Inflammatory and Immune Support from Lycopene and Vitamins C & A. Lycopene's anti-inflammatory properties complement turmeric, holy basil, and reishi, providing a multi-faceted approach to reducing inflammation. Vitamins C and A in tomatoes bolster immune function, working synergistically with adaptogenic mushrooms like turkey tail and reishi, known for their immune-enhancing effects.

(xi) Closing Paragraphs. As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant decrease in protein and/or fiber concentration within the food product, as described herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. An adaptogenic, high-protein (AHP) sauce comprising:
a protein mixture at a concentration by weight of 5%-20%,
Rhodiola Rosea at a concentration by weight of 0.03-6%,
a food base at a concentration by weight of 70%-90%, and
an edible acid at a concentration by weight of 0.3%-1.5%,
wherein the concentrations do not exceed 100%; and
wherein the AHP sauce comprises a protein content of at least 1%.

2. The AHP sauce of claim 1, wherein the Rhodiola Rosea comprises 0.05-5 weight % of the AHP sauce.

3. A composition comprising:
a protein mixture at a concentration by weight of 1-25%,
Rhodiola Rosea at a concentration by weight of 0.01-7%,
a food base at a concentration by weight of 50-99%, and
an edible acid at a concentration by weight of 0-2%;
wherein the concentrations do not exceed 100%.

4. The composition of claim 3, wherein the wherein the Rhodiola Rosea comprises a concentration by weight of 0.03-6% of the composition.

5. The composition of claim 3, wherein the Rhodiola Rosea comprises a concentration by weight of 0.05-5% of the composition.

6. The composition of claim 3, wherein the protein mixture comprises a collagen peptide, a whey protein, and a bone broth protein.

7. The composition of claim 3, wherein the protein mixture comprises:
a collagen peptide at a concentration by weight of 0-25%,
a whey protein at a concentration by weight of 0-20%, and
a bone broth protein at a concentration by weight of 0-20%,
wherein the concentrations do not exceed 25% of the composition.

8. The composition of claim 3, wherein the food base comprises pasta sauce, taco sauce, barbecue sauce, soup base, chili sauce, hot sauce, curry, masala, chutney, or salsa.

9. The composition of claim 3, wherein the edible acid is selected from the group consisting of citric acid, vinegar, malic acid, tartaric acid, acetic acid, benzoic acid, butyric acid, lactic acid, fumaric acid, succinic acid, ascorbic acid, adipic acid, phosphoric acid, and mixtures thereof.

10. The composition of claim 3, further comprising a taste-masking agent at a concentration by weight of less than 1%, less than 0.1%, less than 0.01%, or less than 0.001%.

11. The composition of claim 3, wherein the protein mixture comprises a protein grade of A, A+, or A++.

12. The composition of claim 3, wherein the composition does not comprise added sugar.

13. The composition of claim 3, wherein the composition does not comprise synthetic ingredients.

14. A method of producing an adaptogenic, high-protein (AHP) sauce comprising:
performing an early-stage cooking process by adding early-stage ingredients to a composition;
performing a mid-stage cooking process by adding mid-stage ingredients to the composition;
performing a late-stage cooking process by adding late-stage ingredients to the composition;
adjusting a pH of the AHP sauce; and
packaging the composition, wherein the packaged composition comprises:
a) 1-25 weight % of a protein mixture,
b) 0.01-7 weight % of Rhodiola Rosea, and
c) 50-99 weight % of a food base, wherein the weight % do not exceed 100%;
adjusting a pH of the AHP sauce; and
packaging the composition.

15. The method of claim 14, wherein the Rhodiola Rosea comprises 0.03-6 weight % of the composition.

16. The method of claim 14, wherein the early-stage ingredients comprise the protein mixture or the food base.

17. The method of claim 14, wherein the mid-stage ingredients comprise the protein mixture or the food base.

18. The method of claim 14, wherein the late-stage ingredients comprise Rhodiola Rosea, the protein mixture, or the food base.

19. The method of claim 14, wherein the adjusting the pH comprises adjusting the pH to between 3.0 and 4.8.

20. The method of claim 14, wherein the adjusting the pH comprises adding 0.1-2 weight % of an edible acid.
